SEQUENCE LISTING (12 sequences)

SEQ ID NO: 1 - DNA, 1089 bp, Homo sapiens
  CCXCKR2 (CXCR7, hRDC1) G-protein coupled receptor (GPCR)
atggatctgcatctcttcgactactcagagccagggaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcggcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactctgccttggagcagagcaccaaatga

SEQ ID NO: 2 - protein, 362 aa, Homo sapiens
  CCXCKR2 (CXCR7, hRDC1) G-protein coupled receptor (GPCR)
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 3 - DNA, 1089 bp, Homo sapiens
  CCXCKR2.2 (CXCR7, hRDC1) G-protein coupled receptor (GPCR)
atggatctgcacctcttcgactacgccgagccaggcaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcagcggcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtgtcggagacggagtactccgccttggagcaaaacgccaagtga

SEQ ID NO: 4 - protein, 362 aa, Homo sapiens
  CCXCKR2.2 (CXCR7, hRDC1) G-protein coupled receptor (GPCR)
MDLHLFDYAEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFSGIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQNAK

SEQ ID NO: 5 - DNA, 1089 bp, Homo sapiens
  CCXCKR2.3 (CXCR7, hRDC1) G-protein coupled receptor (GPCR)
atggatctgcatctcttcgactactcagagccagggaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcggcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattgtcgctgtcttctacttcctgctggccagagccatctcggcgtccagtgaccaggagaagcacagcagccggaagatcatcttctcctacgtggtggtcttccttgtctgctggttgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactctgccttggagcagagcaccaaatga

SEQ ID NO: 6 - protein, 362 aa, Homo sapiens
  CCXCKR2.3 (CXCR7, hRDC1) G-protein coupled receptor (GPCR)
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIVAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 7 - DNA, 1089 bp, Homo sapiens; identical to SEQ ID NO: 1
  CCXCKR2.4 (CXCR7, hRDC1) G-protein coupled receptor (GPCR)

SEQ ID NO: 8 - protein, 362 aa, Homo sapiens; identical to SEQ ID NO: 2
  CCXCKR2.4 (CXCR7, hRDC1) G-protein coupled receptor (GPCR)

SEQ ID NO: 9 - DNA, 1089 bp, Homo sapiens
  CCXCKR2.5 (CXCR7, hRDC1) G-protein coupled receptor (GPCR)
atggatctgcatctcttcgactactcagagccagggaacttctcggacatcagctggccgtgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcagcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggttgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactccgccttggagcagagcaccaaatga

SEQ ID NO: 10 - protein, 362 aa, Homo sapiens [sequence omitted]

SEQ ID NO: 11 - DNA, 35 bp, Artificial Sequence
  PCR primer hRDC1F
gaatgcggccgctatggatctgcatctcttcgact

SEQ ID NO: 12 - DNA, 34 bp, Artificial Sequence
  PCR primer hRDC1R
gaatgcggccgctcatttggtgctctgctccaag

(19) United States
(12) Patent Application Publication
(10) Pub. No.: US 2007/0021484 A1
(43) Pub. Date: Jan. 25, 2007

(54) Substituted N-cinnamyl benzamides

(75) Inventors: Anita Melikian, San Francisco, CA (US); John J. Kim Wright, Redwood City, CA (US); Antoni Krasinski, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, Inc., Mountain View, CA (US)

(21) Appl. No.: 11/478,456

(22) Filed: Jun. 28, 2006

Related U.S. Application Data
(60) Provisional application No. 60/695,717, filed on Jun. 29, 2005.

(51) Int. Cl. A61K 31/4172; A61K 31/40; C07D 233/61; C07D 207/30

(52) U.S. Cl. 514/400; 514/408; 548/335.5; 548/561

(57) ABSTRACT

Substituted benzamide compounds are provided along with methods for the use of those compounds for treating cancer.

### CROSS-REFERENCES TO RELATED APPLICATIONS

This application claims priority to U.S. Provisional Application No. 60/695,717, filed on Jun. 29, 2005, which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with support from the U.S. Government under Grant (or contract) No. 1 U19 AI056690-01, awarded by the National Institutes of Health. The government has certain rights in this invention.

### REFERENCE TO A “SEQUENCE LISTING,” A TABLE, OR A COMPUTER PROGRAM LISTING APPENDIX SUBMITTED ON A COMPACT DISK

Attached below.

### BACKGROUND OF THE INVENTION

The present invention is directed to novel compounds and pharmaceutical compositions that inhibit the binding of the SDF-1 chemokine (also known as the CXCL12 chemokine) or I-TAC (also known as CXCL11) to the chemokine receptor CCXCKR2. These compounds are useful in preventing tumor cell proliferation, tumor formation, metastasis, and inflammatory diseases (see also, co-pending U.S. Ser. Nos. 10/912,638 and 11/050,345).

Chemokines are a superfamily of small, cytokine-like proteins that induce cytoskeletal rearrangement, firm adhesion to endothelial cells, and directional migration and may also effect cell activation and proliferation. Chemokines act in a coordinated fashion with cell surface proteins to direct the specific homing of various subsets of cells to specific anatomical sites.

Early research efforts by a number of groups have indicated a role for the chemokine receptor CXCR4 in metastasis and tumor growth. Muller, et al., “Involvement of Chemokine Receptors in Breast Cancer Metastasis,” Nature, 410:50-56 (2001) demonstrated that breast tumor cells use chemokine-mediated mechanisms, such as those regulating leukocyte trafficking, during the process of metastasis. Tumor cells express a distinct, non-random pattern of functionally active chemokine receptors. Signaling through CXCR4 mediates actin polymerization and pseudopodia formation in breast cancer cells, and induces chemotactic and invasive responses. Additionally, the organs representing the main sites of breast cancer metastasis (such as lymph nodes, bone marrow, and lungs) are the most abundant sources of ligand for the CXCR4 receptor.

Using immunodeficient mice, Muller and colleagues succeeded in reducing the metastasis of injected human breast cancer cells by treating mice with an antibody known to bind CXCR4. Their finding suggests that breast cancer metastasis could be reduced by treating a patient with a CXCR4 antagonist.

Bertolini, et al., “CXCR4 Neutralization, a Novel Therapeutic Approach for Non-Hodgkin's Lymphoma,” Cancer Research, 62:3106-3112 (2002) demonstrated a reduction of tumor volume as well as prolonged survival of immunodeficient mice injected with human lymphoma cells treated with anti-CXCR4 antibodies. They interpreted their finding to mean that tumor volume could be reduced by treating a patient with a CXCR4 antagonist.

More recent studies suggest that another chemokine receptor, CCXCKR2, may also be a potential candidate in the treatment of cancer. CCXCKR2 is preferentially expressed in transformed cells over normal cells, with detectable expression in a number of human cancers. In vitro studies indicate that proliferation of CCXCKR2 expressing cells can be inhibited by an antagonist of CCXCKR2. In vivo studies in mice indicate that CCXCKR2 antagonists can inhibit tumor formation and tumor growth.

The potential importance of CCXCKR2 is illustrated by an alternative interpretation of the reduction in tumor volume seen by Bertolini and colleagues. This reduction could clearly be the result of an antibody-mediated clearance, and not the result of the anti-CXCR4 antibody as originally believed. In an antibody-mediated clearance, any antibody that recognized a protein on the cell surface of the lymphoma cells would have had the same effect as that attributed to the anti-CXCR4 antibody. Unfortunately, Bertolini and colleagues studies are inconclusive as to whether the observed tumor response was due to antibody-mediated clearance or interaction with CXCR4.

However it is now known that the lymphoma cells used by Bertolini and colleagues express both CXCR4 and CCXCKR2. SDF-1 is the only ligand for CXCR4. SDF-1 and I-TAC both bind CCXCKR2. Using anti-SDF-1 antibody, it has now been shown that antagonists of CCXCKR2 are responsible for the reduction in tumor load and increased survival rate. Because SDF-1 is the only ligand for CXCR4, one would expect neutralization of SDF-1 with anti-SDF-1 antibody would be equivalent to the neutralization of CXCR4 with anti-CXCR4 antibody. However, experiments using an anti-SDF-1 antibody demonstrated only a partial reduction in tumor load and an increased survival rate. As a result, CCXCKR2 is the likely target, as the continued activity appears due to the interactions of the second ligand, I-TAC, with CCXCKR2.

Until recently, the possible importance of CCXCKR2 in tumor cell proliferation, tumor growth, and metastasis was unknown. Now, with recent evidence pointing to the ability of certain CCXCKR2 antagonists to prevent the growth and spread of cancer, and expression patterns indicating a limited tissue distribution for the CCXCKR2 receptor, it would be beneficial to provide compounds that are able to bind specifically to the CCXCKR2 receptor on tumor cells with potentially few side effects.

Moreover, recently it has been discovered that CCXCKR2 can serve as a co-receptor for certain genetically divergent human immunodeficiency virus (HIV) and simian immunodeficiency virus (SIV), in particular for the HIV-2-ROD, an X4-tropic isolate (Shimizu, N. et al., J. Virol., (2000) 74: 619-626; Balabanian, K., et al., J. Biol. Chem., in press; published on Aug. 17, 2005 as Manuscript M508234200).

Still further, SDF-1, has been described to have a role in the mobilization of hematopoietic progenitor cells and stem cells, and in particular of those cells bearing the CXCR4 receptor, to specific hematopoietic tissues including bone marrow (Hattori, K., et al., Blood, (2000) 97:3354-3360; WO 2005/000333, the disclosure of which are incorporated herein by reference). For example, it is known that CD34+ progenitor cells express CXCR4 and require SDF-1 produced by bone marrow stromal cells for chemoattraction and engraftment, and that in vitro, SDF-1 is chemotactic for both CD34+ cells and for progenitor/stem cells. SDF-1 is also an important chemoattractant, signaling via the CXCR4 receptor, for several other more committed progenitors and mature blood cells including T-lymphocytes and monocytes, pro- and pre-B lymphocytes, and megakaryocytes. As mentioned above, SDF-1 is the only ligand for the CXCR4 receptor. SDF-1 and I-TAC are both ligands for CCXCKR2 receptor. More recent studies suggest that the CCXCKR2 receptor may also play a part in stem cell mobilization processes.

In view of the above, it is apparent that compounds that are able to bind specifically to CCXCKR2 receptors may be useful to treating diseases and other biological conditions that may benefit from such interactions. The present invention provides such compounds along with pharmaceutical compositions and related methods for treatment.

### BRIEF SUMMARY OF THE INVENTION

The present invention provides, in one aspect, compounds having a formula selected from the group consisting of formula I, formula II and formula III, provided below, and all pharmaceutically acceptable salts and hydrates thereof.

The compounds provided herein are useful for binding to CCXCKR2 (also referred to as CXCR7), and treating diseases that are dependent, at least in part, on CCXCKR2 activity. Accordingly, the present invention provides in further aspects, compositions containing one or more of the above-noted compounds in admixture with a pharmaceutically acceptable excipient.

In still another aspect, the present invention provides methods for inhibiting the binding of chemokines I-TAC or SDF-1 to a CCXCKR2 receptor, comprising contacting a compound of the formula above, with a cell that expresses the CCXCKR2 receptor for a time sufficient to inhibit the binding of the chemokines to the CCXCKR2 receptor.

In yet another aspect, the present invention provides methods of treating cancer comprising administering to a subject in need of such treatment a therapeutically effective amount of a compound of the above formula, for a period of time sufficient to treat the cancer.

In still another aspect, the present invention provides methods of treating inflammatory diseases comprising administering to a subject in need of such treatment a therapeutically effective amount of a compound of the above formula, for a period of time sufficient to treat the inflammatory disease.

### BRIEF DESCRIPTION OF THE DRAWINGS

Not applicable

### DETAILED DESCRIPTION OF THE INVENTION

I. Abbreviation and Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom. Non-limiting examples of aryl groups include phenyl, naphthyl and biphenyl, while non-limiting examples of heteroaryl groups include 1-pyrrolyl, 2-pyrrolyl, 3-pyrrolyl, 1-pyrazolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, benzopyrazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl or heteroaryl group is attached to an alkyl group (e.g., benzyl, phenethyl, pyridylmethyl and the like).

The term “heterocycle” refers to a saturated or unsaturated non-aromatic cyclic group containing at least one sulfur, nitrogen or oxygen heteroatom. Each heterocycle can be attached at any available ring carbon or heteroatom. Each heterocycle may have one or more rings. When multiple rings are present, they can be fused together or linked covalently. Each heterocycle must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Non-limiting examples of heterocycle groups include pyrrolidine, piperidine, imidazolidine, pyrazolidine, butyrolactam, valerolactam, imidazolidinone, hydantoin, dioxolane, phthalimide, 1,4-dioxane, morpholine, thiomorpholine, thiomorpholine-S,S-dioxide, piperazine, pyran, pyridone, 3-pyrroline, thiopyran, pyrone, tetrahydrofuran, tetrahydrothiophene and the like.

The above terms (e.g., “alkyl,” “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below, while the term “alkyl” and related aliphatic radicals is meant to refer to unsubstituted version, unless indicated to be substituted.

Substituents for the alkyl radicals (including those groups often referred to as alkylene, alkenyl, alkynyl and cycloalkyl) can be a variety of groups selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂ in a number ranging from zero to (2 m′+1), where m′ is the total number of carbon atoms in such radical. R′, R″ and R′″ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl.

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

“CCXCKR2” also referred to as “RDC1,” refers to a seven-transmembrane domain presumed G-protein coupled receptor (GPCR). The CCXCKR2 dog ortholog was originally identified in 1991. See, Libert et al. Science 244:569-572 (1989). The dog sequence is described in Libert et al., Nuc. Acids Res. 18(7):1917 (1990). The mouse sequence is described in, e.g., Heesen et al., Immunogenetics 47:364-370 (1998). The human sequence is described in, e.g., Sreedharan et al., Proc. Natl. Acad. Sci. USA 88:4986-4990 (1991), which mistakenly described the protein as a receptor of vasoactive intestinal peptide. “CCXCKR2” includes sequences that are substantially similar to or conservatively modified variants of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

II. General

Compounds of the present invention can inhibit the binding of ligands to the CCXCKR2 receptor and are useful in the treatment of cancer, particularly solid tumor cancers and lymphomas. More recently, the inhibition of ligand binding to CCXCKR2 was noted to reduce the severity of rheumatoid arthritis in an animal model.

III. Embodiments of the Invention

Compounds

In one aspect, the present invention provides compounds having formula I, formula II or formula III: 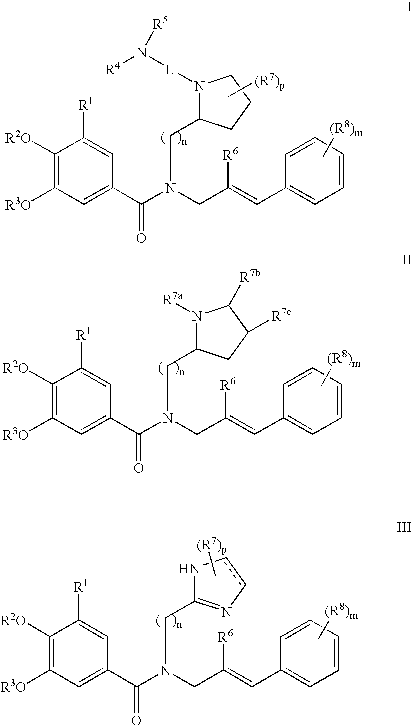
and all pharmaceutically acceptable salts and hydrates thereof.

In the above formulae, the subscript m is an integer of from 0 to 3; the subscript n is an integer of from 1 to 3; the subscript p is an integer of from 0 to 3; and the dotted line of formula III indicates the presence of an optional double bond.

Turning now to the various components of formulae I-III, the letter L represents a C₁₋₄ alkylC₃₋₆ cycloalkyl linking group.

The symbol R¹ represents a member selected from hydrogen, halogen, C₁₋₈ alkoxy, C₁₋₈ alkyl, C₁₋₈ haloalkyl, C₃₋₆ cycloalkyl, C₃₋₆ cycloalkoxy, C₃₋₆ cycloalkyl C₁₋₄ alkyl and C₃₋₆ cycloalkyl C₁₋₄ alkoxy. The symbols R² and R³ each represent members independently selected from C₁₋₈ alkyl and C₁₋₈ haloalkyl, or are optionally combined with the oxygen atoms to which each is attached to from a five- to ten-membered ring.

The symbols R⁴ and R⁵ each independently represent H, C₁₋₈ alkyl, C₁₋₈ haloalkyl, C₃₋₆ cycloalkyl, —COR^{a}, —CO₂R^{a}, —CONR^{a}R^{b}, —SO₂R^{a} or —SO₂NR^{a}R^{b}.

R⁶ represents H or C₁₋₈ alkyl.

Each R⁷ substituent is independently selected from hydrogen, C₁₋₈ alkyl, C₁₋₈ haloalkyl, C₃₋₆ cycloalkyl, C₂₋₈ alkenyl, C₂₋₈ alkynyl, —OR^{a}, —NR^{a}R^{b}, —COR^{a}, —CO₂R^{a}, —CONR^{a}R^{b}, —NR^{a}COR^{b}, —SO₂R^{a}, —X¹COR^{a}, —X¹CO₂R^{a}, —X¹CONR^{a}R^{b}, —X¹NR^{a}COR^{b}, —X¹SO₂R^{a}, —X¹SO₂NR^{a}R^{b}, —X¹NR^{a}R^{b} and —X¹OR^{a}.

Two adjacent members of R^{7a}, R^{7b} and R^{7c} are combined to form a fused five or six-membered ring that is carbocyclic or heterocyclic and optionally substituted with from one to three substituents; and the remaining member of R^{7a} and R^{7c} is R⁷.

Each R⁸ is independently selected from halogen, C₁₋₈ alkyl, C₁₋₈ haloalkyl, C₃₋₆ cycloalkyl, C₂₋₈ alkenyl, C₂₋₈ alkynyl, —OR^{a}, —NR^{a}R^{b}, —COR^{a}, —CO₂R^{a}, —CONR^{a}R^{b}, —NR^{a}COR^{b}, —SO₂R^{a}, —X¹COR^{a}, —X¹CO₂R^{a}, —X¹CONR^{a}R^{b}, —X¹NR^{a}COR^{b}, —X¹SO₂R^{a}, —X¹SO₂NR^{a}R^{b}, —X¹NR^{a}R^{b} and —X¹OR^{a}.

Within the above, each X¹ is selected from C₁₋₄ alkylene and C₂₋₄ alkenylene; and each R^{a} and R^{b} is independently selected from hydrogen, C₁₋₈ alkyl, C₁₋₈ haloalkyl, C₃₋₆ cycloalkyl and aryl-C₁₋₄ alkyl; and the aliphatic portions of each of said R⁷ substituents and the ring formed by combining R^{7a} with R^{7b} or by combining R^{7b} with R^{7c} is optionally substituted with from one to three members selected from the group consisting of —OH, —OR^{m}, —OC(O)NHR^{m}, —OC(O)N(R^{m})₂, —SH, —SR^{m}, —S(O)R^{m}, —S(O)₂R^{m}, —SO₂NH₂, —S(O)₂NHR^{m}, —S(O)₂N(R^{m})₂, —NHS(O)₂R^{m}, —NR^{m}S(O)₂R^{m}, —C(O)NH₂, —C(O)NHR^{m}, —C(O)N(R^{m})₂, —C(O)R^{m}, —NHC(O)R^{m}, —NR^{m}C(O)R^{m}, —NHC(O)NH₂, —NR^{m}C(O)NH₂, —NR^{m}C(O)NHR^{m}, —NHC(O)NHR^{m}, —NR^{m}C(O)N(R^{m})₂, —NHC(O)N(R^{m})₂, —CO₂H, —CO₂R^{m}, —NHCO₂R^{m}, —NR^{m}CO₂R^{m}, —CN, —NO₂, —NH₂, —NHR^{m}, —N(R^{m})₂, —NR^{m}S(O)NH₂ and —NR^{m}S(O)₂NHR^{m}, wherein each R^{m} is independently an unsubstituted C₁₋₆ alkyl.

In one group of embodiments, the compound has formula I. Within this group of embodiments, preferred are those in which m is 2, n is 1 and p is 0. Turning next to the linking group L, preferred linking groups are selected from: 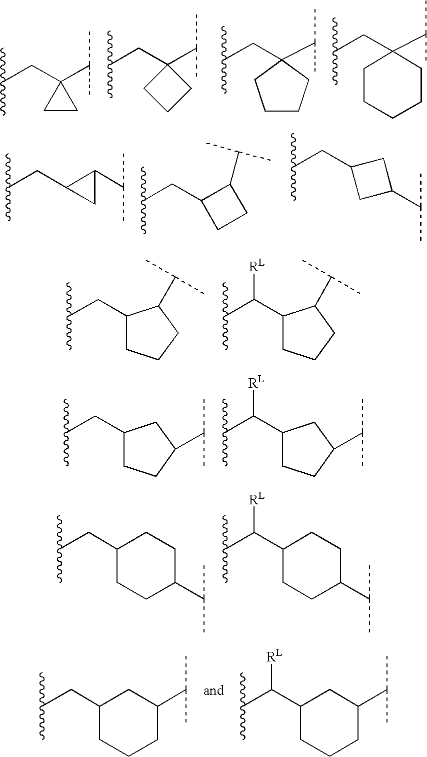
wherein the wavy line indicates the point of attachment to the pyrrolidinyl nitrogen atom, the dashed line indicates the point of attachment to NR⁴R⁵, and R^{L} is a C₁₋₃ alkyl group. Here, the R^{L} moiety represents a vestige of the C₁₋₄alkyl portion of the C₁₋₄alkyl C₃₋₆ cycloalkyl linking group. Still further preferred are those embodiments wherein L is selected from:

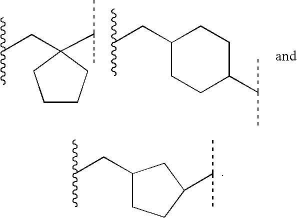

Certain other embodiments of formula I are also preferred. In one group of embodiments, R¹ is H or OCH₃; R² and R³ are each independently selected from the group consisting of C₁₋₃ alkyl and C₁₋₃ haloalkyl; R⁴ and R⁵ are each independently selected from the group consisting of H, C₁₋₄ alkyl, C₁₋₄ haloalkyl, C₃₋₆ cycloalkyl, —COR^{a}, and —SO₂R^{a}; R⁶ is H or CH₃; and each R⁸ when present is independently selected from the group consisting of halogen and C₁₋₄ alkyl. Still further preferred are those embodiments in which R¹ is H or OCH₃; R² and R³ are each independently selected from C₁₋₃ alkyl and C₁₋₃ haloalkyl; R⁴ and R⁵ are each independently selected from H, C₁₋₄ alkyl, C₁₋₄ haloalkyl, C₃₋₆ cycloalkyl, —COR^{a}, and —SO₂R^{a}; R⁶ is H or CH₃; and each R⁸ when present is independently selected from halogen and C₁₋₄ alkyl.

Another group of embodiments are those compounds represented by formula II. In this group of embodiments, one select group are those compounds wherein R^{7b} and R^{7c} are combined to form a five or six-membered ring fused to the pyrrolidine ring. Another select group are those compounds wherein R^{7a} and R^{7b} are combined to form a five or six-membered ring fused to the pyrrolidine ring. In yet another select group are those compounds in which R^{7a} is hydrogen or C₁₋₈ alkyl. Still other preferred embodiments are those compounds in which n is 1 or 2; those compounds in which R¹ is selected from hydrogen and C₁₋₈ alkoxy; and those compounds in which R² and R³ are each independently selected from methyl, ethyl, propyl, isopropyl, butyl, sec-butyl, isobutyl, tert-butyl and C₁₋₄ haloalkyl. A particularly preferred group of embodiments are those compounds in which n is 1 or 2; R¹ is selected from hydrogen and C₁₋₈ alkoxy; and R² and R³ are each independently selected from methyl, ethyl, propyl, isopropyl, butyl, sec-butyl, isobutyl, tert-butyl and C₁₋₄ haloalkyl.

In still another group of embodiments, the compounds are represented by formula III. In certain preferred embodiments of formula III, n is 1 or 2. In other preferred embodiments, R¹ is selected from hydrogen and C₁₋₈ alkoxy. In still other preferred embodiments, R² and R³ are each independently selected from methyl, ethyl, propyl, isopropyl, butyl, sec-butyl, isobutyl, tert-butyl and C₁₋₄haloalkyl. In other preferred embodiments of formula III, n is 1 or 2; R¹ is selected from hydrogen and C₁₋₈ alkoxy; R² and R³ are each independently selected from methyl, ethyl, propyl, isopropyl, butyl, sec-butyl, isobutyl, tert-butyl and C₁₋₄ haloalkyl. In still other preferred embodiments of formula III, m is 1 or 2 and each R⁸ is independently selected from halogen and C₁₋₈ alkyl. In other preferred embodiments of formula III, R⁶ is H or CH₃. In one group of further preferred embodiments, n is 1 or 2; R¹ is selected from hydrogen and C₁₋₈ alkoxy; R² and R³ are each independently selected from methyl, ethyl, propyl, isopropyl, butyl, sec-butyl, isobutyl, tert-butyl and C₁₋₄ haloalkyl; R⁶ is H or CH₃; m is 1 or 2 and each R⁸ is independently selected from halogen and C₁₋₈ alkyl.

Preparation of Substituted N-Cinnamyl Benzamides

The compounds of the present invention can be prepared according to the Examples provided below, including variations on those Examples which would be apparent to one skilled in the art and according to the synthetic procedures outlined below. In the Schemes presented below, the substituent, P, represents a protecting group, the substituent, X, represents a halogen or other leaving group, such as a tosylate, an suitable acyloxy group, and the like, and the remaining substituents, e.g., R¹, R⁷, etc., are as described in the “Compounds” section of the application.

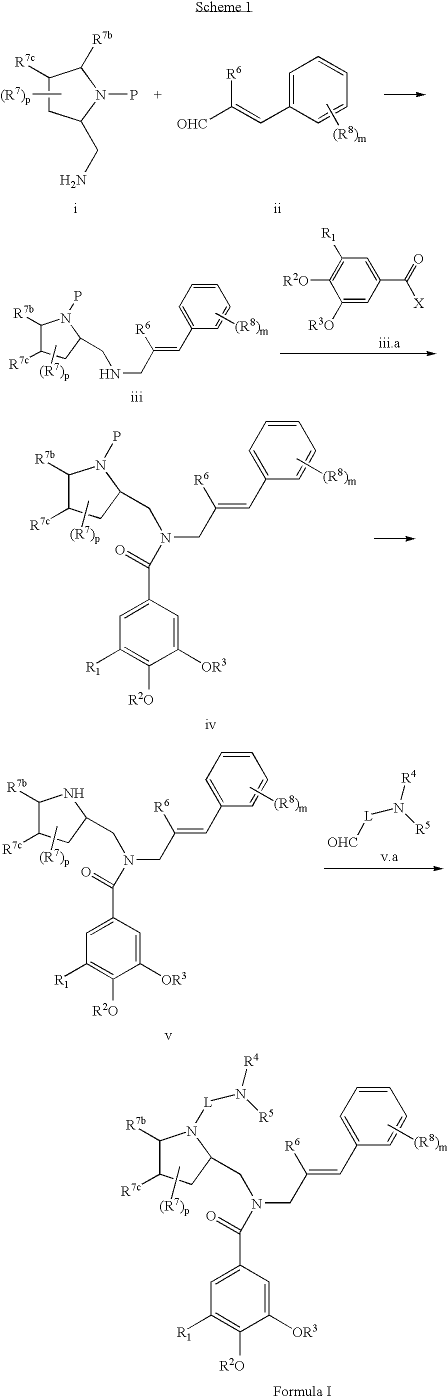

Compounds of formula I can be prepared as described in Scheme I. As shown therein, aldehyde (ii) can undergo a reductive amination reaction with primary amine (i) to form compound (iii). The primary amines (i) can be synthesized by chemical routes known to those of ordinary skill in the art. The reductive amination reaction may be carried out in the presence of a reducing agent in any suitable solvent, including, but not limited to tetrahydrofuran (THF), dichloromethane, or methanol to form the intermediate (iii). Suitable reducing agents include, but are not limited to, sodium cyanoborohydride (see, Mattson, et al., J. Org. Chem. 1990, 55, 2552, and Barney, et al., Tetrahedron Lett. 1990, 31, 5547); sodium triacethoxyborohydride (see, Abdel-Magid, et al., Tetrahedron Lett. 1990, 31, 5595); sodium borohydride (see, Gribble, G. W., et al. Synthesis. 1987, 709); iron pentacarbonyl and alcoholic KOH (see, Watabane, et al., Tetrahedron Lett. 1974, 1879); and BH₃ pyridine (see, Pelter, et al., J. Chem. Soc., Perkin Trans. 1, 1984, 717).

Acylation of compound (iii) with a benzoyl group (iii.a) can produce the acylated product (iv). The acylation reaction can be performed by combining iii with a substituted benzoyl group (iii.a) and a base in any suitable solvent, such as tetrahydrofuran or dichloromethane. Preferred bases include tertiary amine bases, among others. Especially preferred bases include triethylamine and Hunig's base.

Alternatively, acylation of compound iii with a benzoyl group (iii.a), such as a suitably substituted benzoyl halide, to produce compound iv can also be achieved using a suitable coupling reagent, such as propane phosphonic acid cyclic anhydride, O-(benzotriazol-1-yl)-N,N,N′,N′-tetramethyluronium tetrafluoroborate, 1-ethyl-3-(3-dimethylbutylpropyl)carbodiimide or dicyclohexyl-carbodiimide (see, B. Neises and W. Steglich, Angew. Chem., Int. Ed. Engl., 17, 522, 1978), in the presence of a catalyst, such as 4-N,N-dimethylamino-pyridine, or in the presence of hydroxybenzotriazole (see, K. Horiki, Synth. Commun., 7, 251).

Removal of the protecting group, P, in compound iv to produce the secondary amine (v) can be achieved by following chemical routes known to those of ordinary skill in the art as described in “Protective Groups in Organic Synthesis,” by Theodora W. Greene, and Peter G. M. Wuts, (Wiley Interscience). Amine v can be reacted with a suitably substituted amino aldehyde group (v.a) under reductive amination conditions as described above (for the synthesis of compound iii) to produce compound of formula I of the invention.

Compounds having formula II of the invention can be prepared following a the synthetic procedure as outlined in Scheme 2 below.

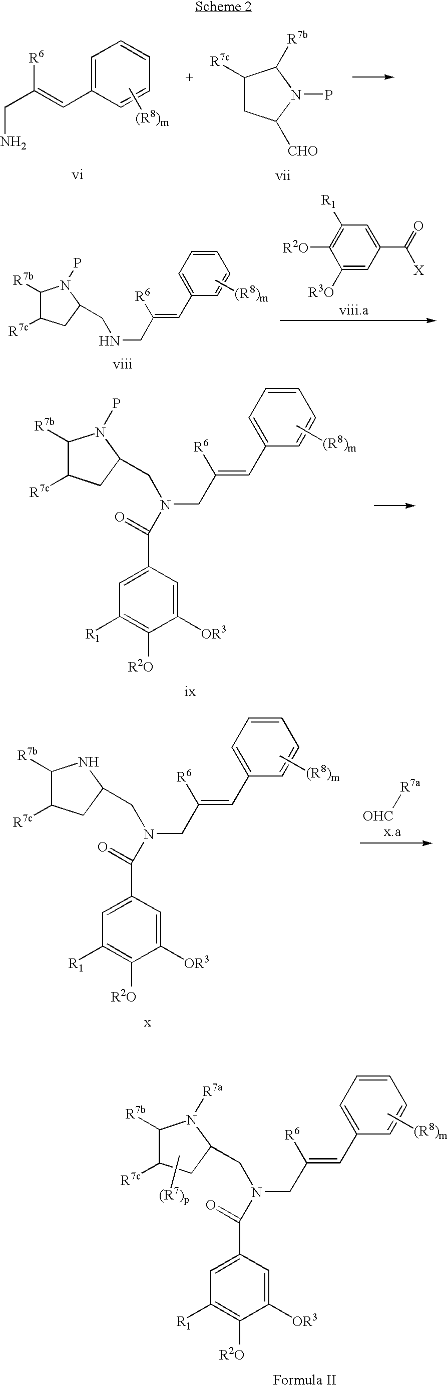

As shown above in Scheme 2, aldehyde (vii) can undergo a reductive amination reaction with primary amine (vi) to form compound (viii). The primary amines (vi) can be synthesized by chemical routes known to those of ordinary skill in the art. The reductive amination reaction may be carried out in the presence of a reducing agent in any suitable solvent, including, but not limited to tetrahydrofuran (THF), dichloromethane, or methanol to form the intermediate (viii). Suitable reducing agents include those as described in Scheme 1.

Acylation of compound (viii) with a benzoyl group (viii.a) can produce the acylated product (ix). The acylation reaction can be performed by combining viii with a substituted benzoyl group (viii.a) and a base in any suitable solvent, such as tetrahydrofuran or dichloromethane. Preferred bases include tertiary amine bases, among others. Especially preferred bases include triethylamine and Hunig's base.

Alternatively, acylation of compound viii with a benzoyl group (viii.a), such as a suitably substituted benzoyl halide, to produce compound ix can also be achieved using a suitable coupling reagent, such as propane phosphonic acid cyclic anhydride, O-(benzotriazol-1-yl)-N,N,N′,N′-tetramethyluronium tetrafluoroborate, 1-ethyl-3-(3-dimethylbutylpropyl)carbodiimide or dicyclohexyl-carbodiimide (see, B. Neises and W. Steglich, Angew. Chem., Int. Ed. Engl., 17, 522, 1978), in the presence of a catalyst, such as 4-N,N-dimethylamino-pyridine, or in the presence of hydroxybenzotriazole (see, K. Horiki, Synth. Commun., 7, 251).

Removal of the protecting group, P, in compound ix to produce the secondary amine (x) can be achieved by following chemical routes known to those of ordinary skill in the art as described in “Protective Groups in Organic Synthesis,” by Theodora W. Greene, and Peter G. M. Wuts, (Wiley Interscience). Amine x can be reacted with a suitably substituted amino aldehyde group (x.a) under reductive amination conditions as described above (for the synthesis of compound viii) to produce compound of formula II of the invention.

Compounds of formula III can be prepared as described below in Scheme 3.

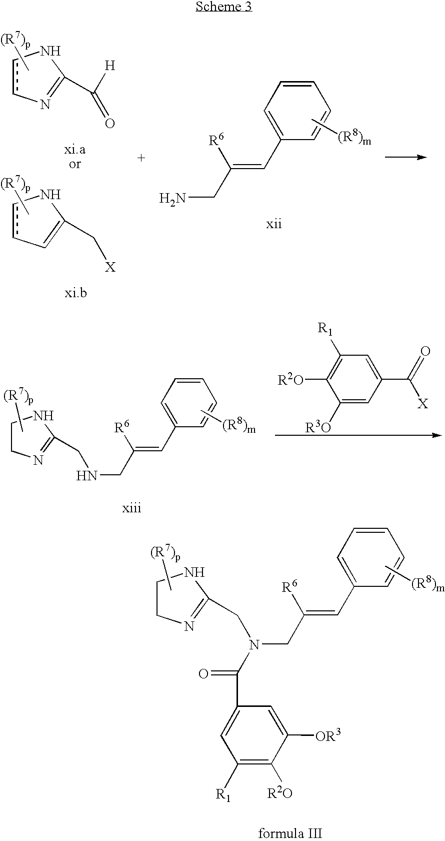

The dialkylamine compound xiii can be prepared by either reductive amination of aldehyde xi.a with amine xii using the synthetic procedures as already described in Schemes 1 and 2. Alternatively, amine xii is alkylated with an appropriately substituted alkylating group (xi.b) to product compound dialkylamine xiii. Suitable reaction conditions for carrying out the alkylation reaction include dissolving xi.b and xii in a solvent such as dimethylformamide, acetonitrile and the like, and adding to the reaction mixture a tertiary amine base such as triethylamine, Hunig's base; or an inorganic base such as potassium carbonate, sodium carbonate (see, March's Advanced Organic Chemistry: Reactions, Mechanisms, and Structure, 5th Edition, by Michael B. Smith and Jerry March (Wiley Inter-Science)). Compound xiii can be further converted to the acylated product, i.e., compound of formula III, using the synthetic procedures as outlined in Schemes 1 and 2.

Compositions

In addition to the compounds provided above, compositions are provided in the present invention that are useful for treating cancer, as well as other diseases modulated by CCXCKR2 in humans and animals. The compositions will typically contain a pharmaceutical carrier or diluent.

The term “composition” as used herein is intended to encompass a product comprising the specified ingredients, preferably in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy and drug delivery. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self emulsifications as described in U.S. patent application Ser. No. 20020012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present invention are employed. As used herein, topical application is also meant to include the use of mouth washes and gargles.

Methods of Use

While not wishing to be bound by any particular theory, the compositions of the present invention are considered to provide a therapeutic effect by inhibiting the binding of SDF-1 and/or I-TAC to the CCXCKR2 receptor. Therefore, the compounds and compositions of the present invention can be used in the treatment or prevention of diseases or disorders in a mammal in which the inhibition of binding of SDF-1 and/or I-TAC to the CCKCR2 receptor would provide a therapeutic effect. Diseases and disorders that can be treated by the compounds or compositions of the present invention include cancer, inflammation, HIV infectivity, progenitor/stem cell disorders, among others. In particular, SDF-1 is known to provide a target for interfering with the development or spread of cancer cells in a mammal, such as a human. Inhibition of the binding of I-TAC to the CCXCKR2 receptor prevents the formation of vascularized tumors. By contacting the compositions described above with a cancer cell that expresses the CCXCKR2 receptor, the response that would otherwise trigger in the cancer cell can be reduced. Accordingly, the present invention is also directed to methods that are useful in the prevention and/or treatment of various disease, including cancer, particularly solid tumor cancers, more particularly breast cancer.

As determined by radiolabeled SDF-1 binding and I-TAC displacement, CCXCKR2 was preferentially expressed in human transformed cells. Included in Table A are those tissue types in which CCXCKR2 was expressed (CCXCKR2⁺) as well as those tissue types in which CCXCKR2 was not expressed (CCXCKR2⁻).

TABLE A

| CCXCKR2⁺ | CCXCKR2⁻ |
|---|---|
| Human Cervical Adenocarcinoma | Normal Mouse Adult Progenitors |
| | (c-kit+ & CD34+ BM derived) |
| Human Adenocarcinoma, Mammary | Human Acute Lymphoblastic |
| Gland | Leukemia, T Cell |
| Human Burkitt's Lymphoma, B | Normal Murine Bone Marrow |
| Lymphocyte | |
| Human Glioblastoma Multiforme, | Normal Murine Thymus |
| Brain | |
| Human Carcinoma, Prostate | Normal Murine Lung |
| Murine Lymphoblastic Leukemia, | Normal Murine Spleen |
| B Lymphocyte | |
| Murine Mammary Gland Tumor | Normal Murine Liver |
| Normal Murine Fetal Liver | Normal Murine PBL |
| Normal Mouse Brain | Normal Human PBL |
| Normal Mouse Kidney | Normal Murine Heart |
| | Normal Murine Pancreas |

In one embodiment, a preferred method of inhibiting the binding of the chemokines SDF-1 and/or I-TAC to a CCXCKR2 receptor includes contacting one or more of the previously mentioned compounds with a cell that expresses the CCXCKR2 receptor for a time sufficient to inhibit the binding of these chemokines to the CCXCKR2 receptor.

Methods of Treating Cancer

More specifically, the present invention also provides a method of treating cancer. A preferred method of treating cancer, includes administering a therapeutically effective amount of one or more of the previously mentioned compounds (or salts thereof) to a cancer patient for a time sufficient to treat the cancer.

For treatment, the compositions of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Standard in vivo assays demonstrating that the compositions of the present invention are useful for treating cancer include those described in Bertolini, F., et al., Endostatin, an antiangiogenic drug, induces tumor stabilization after chemotherapy or anti-CD20 therapy in a NOD/SCID mouse model of human high-grade non-Hodgkin lymphoma. Blood, No. 1, Vol. 96, pp. 282-87 (1 Jul. 2000); Pengnian, L., Antiangiogenic gene therapy targeting the endothelium-specific receptor tyrosine kinase Tie2. Proc. Natl. Acad. Sci. USA, Vol. 95, pp. 8829-34 (July 1998); and Pulaski, B. Cooperativity of Staphylococcal aureus Enterotoxin B Superantigen, Major Histocompatibility Complex Class II, and CD80 for Immunotherapy of Advanced Spontaneous Metastases in a Clinically Relevant Postoperative Mouse Breast Cancer Model. Cancer Research, Vol. 60, pp. 2710-15 (May 15, 2000).

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex and diet of the subject, as well as the mode and time of administration, rate of excretion, drug combination, and the severity of the particular condition for the subject undergoing therapy.

The compounds and compositions of the present invention can be combined with other compounds and compositions having related utilities to prevent and treat cancer and diseases or conditions associated with CCXCKR2 signaling. Such other drugs may be administered, by a route and in an amount commonly used therefor, contemporaneously or sequentially with a compound or composition of the present invention. When a compound or composition of the present invention is used contemporaneously with one or more other drugs, a pharmaceutical composition containing such other drugs in addition to the compound or composition of the present invention is preferred. Accordingly, the pharmaceutical compositions of the present invention include those that also contain one or more other active ingredients or therapeutic agents, in addition to a compound or composition of the present invention. Examples of other therapeutic agents that may be combined with a compound or composition of the present invention, either administered separately or in the same pharmaceutical compositions, include, but are not limited to: cisplatin, paclitaxel, methotrexate, cyclophosphamide, ifosfamide, chlorambucil, carmustine, carboplatin, vincristine, vinblastine, thiotepa, lomustine, semustine, 5-fluorouracil and cytarabine. The weight ratio of the compound of the present invention to the second active ingredient may be varied and will depend upon the effective dose of each ingredient. Generally, an effective dose of each will be used. Thus, for example, when a compound of the present invention is combined with a second anticancer agent, the weight ratio of the compound of the present invention to the second agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Methods of Treating Inflammation

Still further, the compounds and compositions of the present invention are useful for the treatment of inflammation, and can be combined with other compounds and compositions having therapeutic utilities that may require treatment either before, after or simultaneously with the treatment of cancer or inflammation with the present compounds. Accordingly, combination methods and compositions are also a component of the present invention to prevent and treat the condition or disease of interest, such as inflammatory or autoimmune disorders, conditions and diseases, including inflammatory bowel disease, rheumatoid arthritis, osteoarthritis, psoriatic arthritis, polyarticular arthritis, multiple sclerosis, allergic diseases, psoriasis, atopic dermatitis and asthma, and those pathologies noted above.

For example, in the treatment or prevention of inflammation or antimmunity or for example arthritis associated bone loss, the present compounds and compositions may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds and compositions may be administered with an analgesic listed above; a potentiator such as caffeine, an H2 antagonist (e.g., ranitidine), simethicone, aluminum or magnesium hydroxide; a decongestant such as phenylephrine, phenylpropanolamine, pseudoephedrine, oxymetazoline, ephinephrine, naphazoline, xylometazoline, propylhexedrine, or levo desoxy ephedrine; an antitussive such as codeine, hydrocodone, caramiphen, carbetapentane, or dextromethorphan; a diuretic; and a sedating or non sedating antihistamine.

As noted, compounds and compositions of the present invention may be used in combination with other drugs that are used in the treatment, prevention, suppression or amelioration of the diseases or conditions for which compounds and compositions of the present invention are useful. Such other drugs may be administered, by a route and in an amount commonly used therefor, contemporaneously or sequentially with a compound or composition of the present invention. When a compound or composition of the present invention is used contemporaneously with one or more other drugs, a pharmaceutical composition containing such other drugs in addition to the compound or composition of the present invention is preferred. Accordingly, the pharmaceutical compositions of the present invention include those that also contain one or more other active ingredients or therapeutic agents, in addition to a compound or composition of the present invention. Examples of other therapeutic agents that may be combined with a compound or composition of the present invention, either administered separately or in the same pharmaceutical compositions, include, but are not limited to: (a) VLA-4 antagonists, (b) corticosteroids, such as beclomethasone, methylprednisolone, betamethasone, prednisone, prenisolone, dexamethasone, fluticasone, hydrocortisone, budesonide, triamcinolone, salmeterol, salmeterol, salbutamol, formeterol; (c) immunosuppressants such as cyclosporine (cyclosporine A, Sandimmune®, Neoral®), tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchloipheniramine, triprolidine, clemastine, diphenhydramine, diphenylpyraline, tripelemlamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non steroidal anti asthmatics (e.g., terbutaline, metaproterenol, fenoterol, isoetharine, albuterol, bitolterol and pirbuterol), theophylline, cromolyn sodium, atropine, ipratropium bromide, leukotriene antagonists (e.g., zafmlukast, montelukast, pranlukast, iralukast, pobilukast and SKB-106,203), leukotriene biosynthesis inhibitors (zileuton, BAY-1005); (f) non steroidal anti-inflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone); (g) cyclooxygenase-2 (COX-2) inhibitors such as celecoxib (Celebrex®) and rofecoxib (Vioxx®); (h) inhibitors of phosphodiesterase type IV (PDE IV); (i) gold compounds such as auranofin and aurothioglucose, (j) etanercept (Enbrel®), (k) antibody therapies such as orthoclone (OKT3), daclizumab (Zenapax®), basiliximab (Simulect®) and infliximab (Remicade®), (l) other antagonists of the chemokine receptors, especially CCR5, CXCR2, CXCR3, CCR2, CCR3, CCR4, CCR7, CX₃CR1 and CXCR6; (m) lubricants or emollients such as petrolatum and lanolin, (n) keratolytic agents (e.g., tazarotene), (o) vitamin D₃ derivatives, e.g., calcipotriene or calcipotriol (Dovonex®), (p) PUVA, (q) anthralin (Drithrocreme®), (r) etretinate (Tegison®) and isotretinoin and (s) multiple sclerosis therapeutic agents such as interferon β-1β (Betaseron®), interferon (β-1α (Avonex(®), azathioprine (Imurek®, Imuran®), glatiramer acetate (Capoxone®), a glucocorticoid (e.g., prednisolone) and cyclophosphamide (t) DMARDS such as methotrexate (u) other compounds such as 5-aminosalicylic acid and prodrugs thereof; hydroxychloroquine; D-penicillamine; antimetabolites such as azathioprine, 6-mercaptopurine and methotrexate; DNA synthesis inhibitors such as hydroxyurea and microtubule disrupters such as colchicine. The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Method of Treating HIV Infectivity

Still further, the compounds and compositions of the present invention are useful for the (prophylactic, curative or palliative) treatment of HIV infectivity, and can be combined with other compounds and compositions having therapeutic utilities that may require treatment either before, after or simultaneously with the treatment of HIV infectivity with the present compounds.

In certain aspects, in the treatment of HIV infectivity, an appropriate dosage level will generally be about 0.001 to 100 mg per kg patient body weight per day which can be administered in single or multiple doses. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Included within the scope of the invention are embodiments comprising the co-administration of a compound of the invention with one or more additional therapeutic agents, and compositions containing a compound of the invention along with one or more additional therapeutic agents. Such a combination therapy is especially useful for the prevention and/or treatment of infection by HIV and related retroviruses which may evolve rapidly into strains resistant to any monotherapy. Alternatively, additional therapeutic agents may be desirable to treat diseases and conditions which result from or accompany the disease being treated with the compound of the invention. For example, in the treatment of an HIV or related retroviral infection, it may be desirable to additionally treat opportunistic infections, neoplasms and other conditions which occur as a result of the immuno-compromised state of the patient being treated.

Preferred combinations of the invention include simultaneous or sequential treatment with a compound of the invention and one or more: (a) reverse transcriptase inhibitors such as abacavir, adefovir, didanosine, lamivudine, stavudine, zalcitabine and zidovudine; (b) non-nucleoside reverse transcriptase inhibitors such as capavirine, delavirdine, efavirenz, and nevirapine; (c) HIV protease inhibitors such as indinivir, nelfinavir, ritonavir, and saquinavir; (d) CCR5 antagonists such as TAK-779 or UK-427,857; (e) CXCR4 antagonists such as AMD-3100; (f) integrase inhibitors, such as L-870,810 or S-1360; (g) inhibitors of viral fusion such as T-20; (h) investigational drugs such as trizivir, KNI-272, amprenavir, GW-33908, FTC, PMPA, MKC-442, MSC-204, MSH-372, DMP450, PNU-140690, ABT-378, KNI-764, DPC-083, TMC-120 or TMC-125; (i) antifungal agents, such as fluconazole, itraconazole or voriconazole; or () antibacterial agents, such as azithromycin.

Method of Treating Progenitor/Stem Cell Mobilization Disorders

Still further, the compounds and compositions of the present invention can be useful for the treatment of progenitor/stem cell differentiation and mobilization disorders using procedures and protocols as described in WO05/000333, incorporated herein by reference in its entirety for all purposes. Typical conditions which may be ameliorated or otherwise benefited include hematopoietic disorders, such as aplastic anemia, leukemias, drug-induced anemias, and hematopoietic deficits from chemotherapy or radiation therapy. Still further, the compounds and compositions of the invention can be used in enhancing the success of transplantation during and following immunosuppressive treatments as well as in effecting more efficient wound healing and treatment of bacterial infections.

In the treatment or prevention of progenitor or stem cell mobilization disorders an appropriate dosage level will generally be about 0.001 to 100 mg per kg patient body weight per day which can be administered in single or multiple doses. The compounds may be administered as a single dose, a dose over time, as in i.v., or transdermal administration, or in multiple doses. The compounds of the invention can also be be used in ex vivo treatment protocols to prepare cell cultures which are then used to replenish the blood cells of the subject. Ex vivo treatment can be conducted on autologous cells harvested from the peripheral blood or bone marrow or from allografts from matched donors.

The present compounds can be combined with other compounds and compositions having therapeutic utilities that may require treatment either before, after or simultaneously with the treatment of the progenitor/stem cell disorder with the present compounds. Accordingly, combination methods and compositions are also a component of the present invention to prevent and treat the condition or disease of interest.

### IV. Examples

### Example 1

This example illustrates the preparation of N-[(E)-3-(2,4-Difluoro-phenyl)-2-methyl-allyl]-3,4-dimethoxy-N-[(2S,3aS,7aS)-1-(octahydro-indol-2-yl)methyl]-benzamide (5)

Step 1: (2S,3aS,7aS)-2-Hydroxymethyl-octahydro-indole-1-carboxylic acid tert-butyl ester

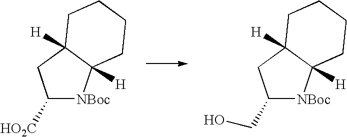

501 mg (1.86 mmol) of (2S,3aS,7aS)-Octahydro-indole-1,2-dicarboxylic acid 1-tert-butyl ester was dissolved in 10 mL methanol, cooled down to 0° C. and a 2M etherous solution of trimethylsilyl diazomethane was added dropwise until the color of the solution became permanently yellow. Excess of trimethylsilyl diazomethane was quenched with a drop of acetic acid and the solution was evaporated. The residue was dissolved in 15 mL THF and 81 mg (3.72 mmol) of lithium borohydride were added at room temperature followed by 3 drops of water. After 1 hour some more lithium borohydride was added and the reaction was complete within the next hour. It was carefully quenched with aqueous sodium bicarbonate. and extracted 3 times with Et₂O. The combined organic layers were dried with anhydrous MgSO₄ and evaporated in vacuum. Flash chromatography using 40-50% ethyl acetate in hexane yielded 432 mg of the product as colorless oil. LC-MSD, m/z for C₁₄H₂₅NO₃ [M+Na]+: 278.1, [2M+Na]+: 533.3

Step 2: (2S,3aS,7aS)-2-Formyl-octahydro-indole-1-carboxylic acid tert-butyl ester

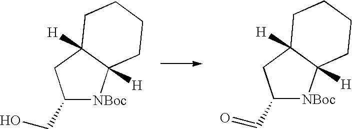

To a solution of 102 mg (0.40 mmol) of (2S,3aS,7aS)-2-Hydroxymethyl-octahydro-indole-1-carboxylic acid tert-butyl ester in 5 mL DCM, 2 mL of 0.2 M Dess-Martin periodinane/DCM was added at r.t. The reaction was stirred for 1 hour at r.t., and then diluted with 20 mL DCM and quenched by the addition of aqueous sodium bicarbonate and sodium sulfite and vigorous stirring for 30 minutes. The organic layer was dried with anhydrous MgSO₄, and evaporated in vacuum to give pale yellow oil. The crude residue was used without purification in the next step. LC-MSD, m/z for C₁₄H₂₃NO₃ [M+Na]+: 276.1, [2M+Na]+: 529.3

Step 3: (2S,3aS,7aS)-2-{[3-(2,4-Difluoro-phenyl)-2-methyl-allylamino]-methyl}-octahydro-indole-1-carboxylic acid tert-butyl ester

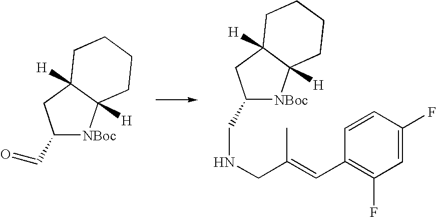

To a solution of 101 mg (0.4 mmol) of (2S,3aS,7aS)-2-Formyl-octahydro-indole-1-carboxylic acid tert-butyl ester in 4 mL DCM, 73 mg (0.4 mmol) of (E)-3-(2,4-Difluoro-phenyl)-2-methyl-allylamine and 1 g activated molecular sieves were added and the mixture was stirred at r.t. for 2 hours. 127 mg (0.6 mmol) of sodium triacetoxyborohydride were added and the mixture was stirred for 3 hours, then diluted with 20 mL DCM and quenched with 15 mL of aqueous sodium bicarbonate. The aqueous layer was extracted once with DCM and the combined organic layers were dried with anhydrous MgSO₄, evaporated in vacuum and purified using reverse phase HPLC, mobile phase with a gradient 25-80% acetonitrile in 50 min. Fractions containing pure product were evaporated, the residue was dissolved in DCM, which was washed with aqueous sodium bicarbonate, dried with anhydrous MgSO₄ and evaporated in vacuo to yield 106 mg of the product as the free base pale yellow oil. LC-MSD, m/z for C₂₄H₃₄F₂N₂O₂ [M+H]+: 421.2

Step 4: N-[(E)-3-(2,4-Difluoro-phenyl)-2-methyl-allyl]-3,4-dimethoxy-N-[(2S,3aS,7aS)-1-(octahydro-indol-2-yl)methyl]-benzamide

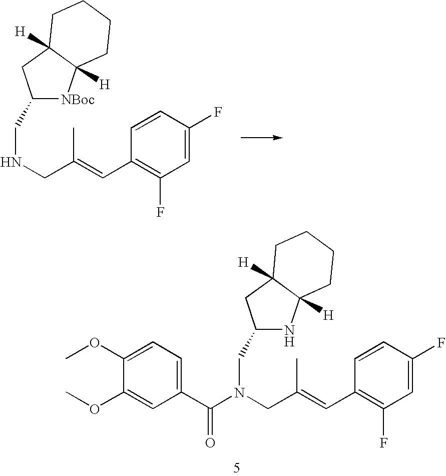

To a solution of 44 mg (0.10 mmol) of (2S,3aS,7aS)-2-{[3-(2,4-Difluoro-phenyl)-2-methyl-allylamino]-methyl}-octahydro-indole-1-carboxylic acid tert-butyl ester in 1 mL DCM, 16 μL of triethylamine (0.12 mmol) and 22 mg (0.11 mmol) of 3,4-dimethoxybenzoyl chloride were added at r.t. Stirring at r.t. for 1 hour was followed by the addition of 1 mL trifluoroacetic acid; 2 hours thereafter the solution was evaporated in vacuum and purified using reverse phase HPLC, mobile phase with a gradient 20-80% acetonitrile in 40 min. Fractions containing pure product were evaporated, the residue was dissolved in DCM, which was washed with aqueous NaHCO₃, dried with anhydrous MgSO₄ and evaporated in vacuo to yield 46 mg of the product as the free base pale yellow viscous oil. LC-MSD, m/z for C₂₈H₃₄F₂N₂O₃ [M+H]+: 485.2; ¹H NMR (400 MHz, CDCl₃/HCl): δ 1.2-1.3 (m, 2H), 1.4-1.5 (m, 4H), 1.6-1.9 (m, 8H), 1.9-2.1 (m, 3H), 2.1-2.3 (m, 1H), 2.5-2.6 (m, 1H), 3.3 (d, 1H), 3.7-3.8 (m, 1H), 3.9 (s, 3H), 3.95 (s, 3H), 4.1-4.2 (m, 1H), 4.3-4.5 (m, 3H), 6.4 (s, 1H), 6.8-6.9 (m, 3H), 7.2-7.4 (in, 5H), 8.1 (bs, 1H), 11.9 (bs, 1H).

### Example 2

### 4-Difluoromethoxy-N-[(E)-3-(2,4-difluoro-phenyl)-2-methyl-allyl]-3-methoxy-N-[(2S,3aS,7aS)-1-(octahydro-indol-2-yl)methyl]-benzamide, (3)

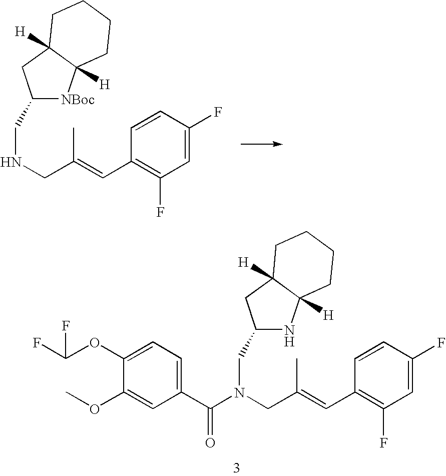

Experimental conditions analogous to described for example 1 (step 4) were used with 29 mg (0.10 mmol) of (2S,3aS,7aS)-2-{[3-(2,4-Difluoro-phenyl)-2-methyl-allylamino]-methyl}-octahydro-indole-1-carboxylic acid tert-butyl ester, 1 mL DCM, 11 μL of triethylamine (0.12 mmol), 18 mg (0.11 mmol) of 4-Difluoromethoxy-3-methoxy-benzoyl chloride. The deprotection was conducted in the same step by addition of 0.5 mL trifluoroacetic acid after 1 h. Product was converted to the hydrochloride salt. Yield 26 mg of pale yellow viscous oil. LC-MSD, m/z for C₂₈H₃₂F₄N₂O₃ [M+H]+: 521.2; ¹H NMR (400 MHz, CDCl₃/HCl): δ 1.2-1.9 (m, 12H), 1.9-2.1 (m, 1H), 2.2-2.3 (m, 1H), 2.5-2.6 (m, 1H), 3.3 (d, 1H), 3.7-3.8 (m, 1H), 3.9 (s, 3H), 4.0-4.2 (m, 2H), 4.3-4.5 (m, 2H), 4.8 (bs, 2H), 6.3 (s, 1H), 6.6 (t, 1H), 6.8-6.9 (m, 2H), 7.1-7.2 (m, 3H), 7.4 (s, 1H), 7.9 (bs, 1H), 11.7 (bs, 1H).

### Example 3

### 7-Methoxy-2,2-dimethyl-benzo[1,3]dioxole-5-carboxylic acid [(E)-3-(2,4-difluoro-phenyl)-2-methyl-allyl]-[(2S,3aS,7aS)-1-(octahydro-indol-2-yl)methyl]-amide, (4)

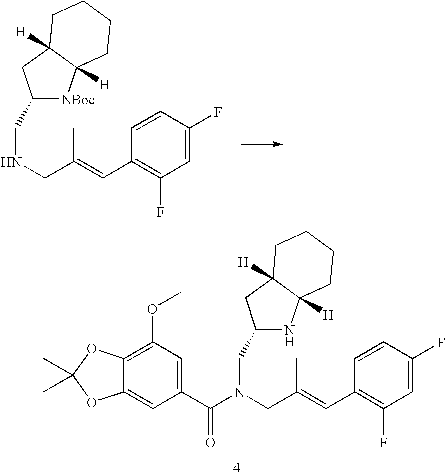

33 mg (0.078 mmol) of (2S,3aS,7aS)-2-{[3-(2,4-Difluoro-phenyl)-2-methyl-allylamino]-methyl}-octahydro-indole-1-carboxylic acid tert-butyl ester, 19 mg (0.086 mmol) of 7-Methoxy-2,2-dimethyl-benzo[1,3]dioxole-5-carboxylic acid and 35 mg (0.12 mmol) of 4-(4,6-dimethoxy[1.3.5]triazin-2-yl)-4-methylmorpholinium chloride hydrate were dissolved in a mixture of 1 mL of DCM and 1 mL of acetonitrile and stirred overnight. The solvents were evaporated and the residue purified on silica using 20% ethyl acetate/hexane. The fractions containing the intermediate product were evaporated and dissolved in a mixture of 5 mL DCM and 0.5 mL trifluoroacetic acid. After 1 hour at r.t. the acid was neutralized with aqueous sodium bicarbonate., the organic layer evaporated and purified using reverse phase HPLC, mobile phase with a gradient 20-80% acetonitrile in 50 min. The fractions containing the product were evaporated, dissolved in DCM and free-based with aqueous sodium bicarbonate. The organic solution was dried with anhydrous MgSO₄ and evaporated in vacuum to yield 20 mg of the product as pale yellow viscous oil. LC-MSD, m/z for C₃₀H₃₆F₂N₂O₄ [M+H]+: 527.2; ¹H NMR (400 MHz, CDCl₃/HCl): δ 1.2-2.0 (m, 18H), 2.2-2.3 (m, 2H), 2.5-2.6 (m, 1H), 3.3 (d, 1H), 3.7-3.8 (m, 1H), 3.9 (s, 3H), 4.1-4.2 (m, 1H), 4.3-4.4 (m, 3H), 5.9 (bs, 1H), 6.3 (s, 1H), 6.7 (s, 1H), 6.8-6.9 (m, 2H), 7.0 (s, 1H), 7.2-7.3 (m, 1H), 7.9 (bs, 1H), 11.9 (bs, 1H).

### Example 4

This example illustrates the preparation of 7-Methoxy-2,2-dimethyl-benzo[1,3]dioxole-5-carboxylic acid (2-methyl-3-phenyl-allyl)-[(2S,6S,7S)-1-(octahydro-indol-2-yl)methyl]-amide (1)

Step 1: (2S,6S,7S)-2-[(2-Methyl-3-phenyl-allylamino)-methyl]-octahydro-indole-1-carboxylic acid tert-butyl ester

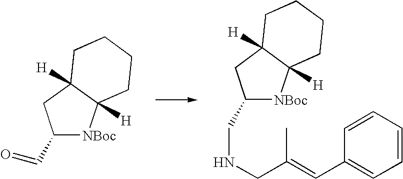

Experimental conditions analogous to described for example 1, step 3 were used with 193 mg (0.76 mmol ) of (2S,3aS,7aS)-2-Formyl-octahydro-indole-1-carboxylic acid tert-butyl ester, 123 mg (0.84 mmol) of (E)-2-Methyl-3-phenyl-allylamine, 5 mL DCM, and 242 mg (1.1 mmol) of sodium triacetoxyborohydride. Product was purified using reverse phase HPLC, mobile phase with a gradient 15-80% acetonitrile in 50 min. Fractions containing pure product were evaporated with 1.5 mL 1M HCl aq. to yield 305 mg of the hydrochloride salt as a yellow oil. LC-MSD, m/z for C₂₄H₃₆N₂O₂ [M+H]+: 385.4

Step 2: 7-Methoxy-2,2-dimethyl-benzo[1,3]dioxole-5-carboxylic acid (2-methyl-3-phenyl-allyl)-[(2S,6S,7S)-1-(octahydro-indol-2-yl)methyl]-amide

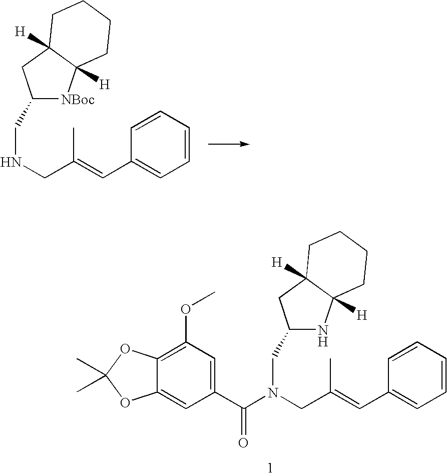

77 mg (0.18 mmol) of (2S,6S,7S)-2-[(2-Methyl-3-phenyl-allylamino)-methyl]-octahydro-indole-1-carboxylic acid tert-butyl ester hydrochloride, 41 mg (0.20 mmol) of 7-Methoxy-2,2-dimethyl-benzo[1,3]dioxole-5-carbonyl chloride and 64 μL (0.45 mmol) of triethylamine were dissolved in a mixture of 3 mL of DCM and stirred for 1 hour, followed by the addition of 0.3 mL trifluoroacetic acid. After 1.5 hours at r.t. the acid was neutralized with aqueous sodium bicarbonate, the organic layer evaporated and purified using reverse phase HPLC, mobile phase with a gradient 15-80% acetonitrile in 50 min. The fractions containing the product were evaporated, dissolved in DCM and free-based with sodium bicarbonate. The organic solution was dried with anhydrous MgSO₄ and evaporated in vacuum to yield 62 mg of the product as pale yellow oil. LC-MSD, m/z for C₃₀H₃₈N₂O₄ [M+H]+: 491.2; ¹H NMR (400 MHz, CDCl₃/HCl): δ 1.2-2.0 (m, 17H), 2.1-2.2 (m, 1H), 2.5-2.6 (m, 1H), 3.3 (d, 1H), 3.7-3.8 (m, 1H), 3.9 (s, 3H), 4.1-4.2 (m, 1H), 4.3-4.4 (m, 3H), 6.4 (s, 1H), 6.7 (s, 1H), 7.0 (s, 1H), 7.1-7.15 (m, 5H), 7.15-7.2 (m, 2H).

### Example 5

This example illustrates the preparation of 7-Methoxy-2,2-dimethyl-benzo[1,3]dioxole-5-carboxylic acid (2-methyl-3-phenyl-allyl)-[(2S,5S,6S)-1-(octahydro-cyclopenta[b]pyrrol-2-yl)methyl]-amide, (2)

Step 1: (2S,5S,6S)-2-Hydroxymethyl-hexahydro-cyclopenta[b]pyrrole-1-carboxylic acid tert-butyl ester

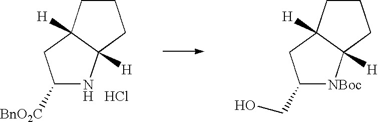

1.09 g (3.87 mmol) of (2S,5S,6S)-Octahydro-cyclopenta[b]pyrrole-2-carboxylic acid benzyl ester hydrochloride was dissolved in 20 mL DCM and 80 mL of aqueous bicarbonate were added along with 930 mg (4.26 mmol) of Boc anhydride. The reaction was stirred at room temperature for 24 hours, then the layers were separated, aqueous layer washed with DCM. The combined organic layers were dried with anhydrous MgSO₄ and evaporated in vacuum. The residue was dissolved in 20 mL THF and 176 mg (8.13 mmol) of lithium borohydride were added at room temperature followed after 30 minutes by 1 mL of water. After 1 hour some more lithium borohydride was added and the reaction was complete within the next hour. It was carefully quenched with sodium bicarbonate and extracted 3 times with Et₂O. The combined organic layers were dried with anhydrous MgSO₄ and evaporated in vacuo. The residue was kept on high vacuum for several days to remove benzyl alcohol. The product was crystallized from DCM/hexane in cold to afford 380 mg of pure crystalline white compound. LC-MSD, m/z for C₁₃H₂₃NO₃ [M+2H−Boc]+: 142.1, [M+Na]+: 264.1, [2M+Na]+: 505.3.

Step 2: (2S,5S,6S)-2-Formyl-hexahydro-cyclopenta[b]pyrrole-1-carboxylic acid tert-butyl ester

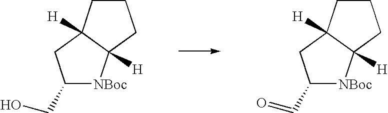

Experimental conditions analogous to described for example 1 step 2 were used with 190 mg (0.79 mmol), of (2S,5S,6S)-2-Hydroxymethyl-hexahydro-cyclopenta[b]pyrrole-1-carboxylic acid tert-butyl ester, 2 mL DCM, and 3.9 mL of 0.2M Dess-Martin periodinane/DCM. Yield 100% of crude product used in next step without purification. LC-MSD, m/z for C₁₃H₂₁NO₃ [M+Na]+: 262.1, [2M+Na]+: 501.3.

Step 3: (2S,5S,6S)-2-[(2-Methyl-3-phenyl-allylamino)-methyl]-hexahydro-cyclopenta[b]pyrrole-1-carboxylic acid tert-butyl ester

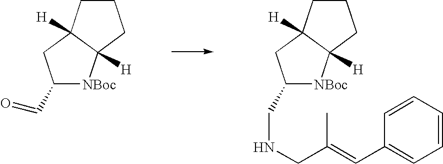

Experimental conditions analogous to described for example 1 (step 3) were used with 188 mg (0.78 mmol) of (2S,5S,6S)-2-Formyl-hexahydro-cyclopenta[b]pyrrole-1-carboxylic acid tert-butyl ester, 126 mg (0.86 mmol) of (E)-2-Methyl-3-phenyl-allylamine, 5 mL DCM, and 248 mg (1.17 mmol) of sodium triacetoxyborohydride. Product was purified using reverse phase HPLC, mobile phase with a gradient 15-80% acetonitrile in 50 min. Fractions containing pure product were evaporated with 1.5 mL 1M HCl aq. to yield 320 mg of the hydrochloride salt as a pale yellow oil. LC-MSD, m/z for C₂₃H₃₄N₂O₂ [M+H]+: 371.4.

Step 4: 7-Methoxy-2,2-dimethyl-benzo[1,3]dioxole-5-carboxylic acid (2-methyl-3-phenyl-allyl)-[(2S,5S,6S)-1-(octahydro-cyclopenta[b]pyrrol-2-yl)methyl]-amide

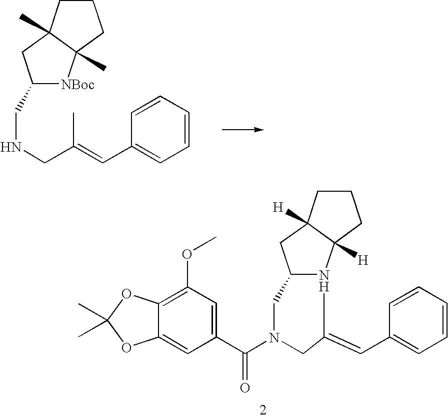

Experimental conditions analogous to described for example 1 (step 4) were used with 74 mg (0.18 mmol) of (2S,5S,6S)-2-[(2-Methyl-3-phenyl-allylamino)-methyl]-hexahydro-cyclopenta[b]pyrrole-1-carboxylic acid tert-butyl ester hydrochloride, 3 mL DCM, 49 mg (0.20 mmol), of 7-Methoxy-2,2-dimethyl-benzo[1,3]dioxole-5-carbonyl chloride, 63 μL (0.45 mmol) of triethylamine and 0.3 mL TFA. Yield: 41 mg of free base as white solid. LC-MSD, m/z for C₂₉H₃₆N₂O₄ [M+H]+: 477.2; ¹H NMR (400 MHz, CDCl₃/HCl): δ 1.2-2.1 (m, 14H), 2.3-2.4 (m, 1H), 2.9-3.0 (m, 1H), 3.7-3.8 (m, 1H), 3.9 (s, 3H), 4.0-4.1 (m, 2H), 4.2-4.3 (m, 3H), 6.4 (s, 1H), 6.7 (s, 1H), 7.0 (s, 1H), 7.1-7.15 (m, 5H), 7.15-7.2 (m, 2H), 8.0 (bs, 1H), 11.6 (bs, 1H).

### Example 6

### N-[(E)-3-(2,4-Difluoro-phenyl)-2-methyl-allyl]-N-(4,5-dihydro-1 H-imidazol-2-ylmethyl)-3,4-dimethoxy-benzamide hydrochloride, (19)

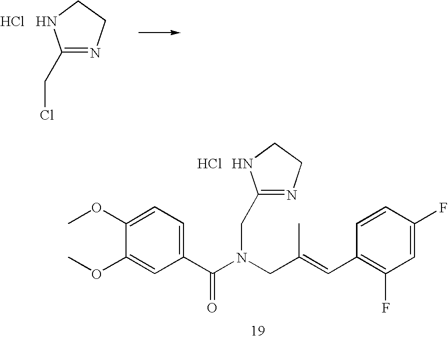

The 2-Chloromethyl-4,5-dihydro-1H-imidazole hydrochloride 53 mg (0.34 mmol), was heated to 70° C. with (E)-3-(2,4-Difluoro-phenyl)-2-methyl-allylamine (0.34 mmol) in 1 mL ethanol for 3 days. The solvent was evaporated and the residue dissolved/suspended in 3 mL DCM followed by the addition of 61 mg (0.31 mmol of 3,4-dimethoxybenzoyl chloride and 91 μL (0.65 mmol) of triethylamine. After 1 hour the solvent was evaporated and the product was purified using reverse phase HPLC, mobile phase with a gradient 15-80% acetonitrile in 50 min. Fractions containing pure product were evaporated with 1 mL 1M HCl aq. to yield 42 mg of the hydrochloride salt as pale yellow solid. LC-MSD, m/z for C₂₃H₂₅F₂N₃O₃ [M+H]+: 430.2; ¹H NMR (400 MHz, CDCl₃/HCl): δ 1.7 (s, 3H), 3.85 (s, 3H), 3.9 (s, 3H), 3.95 (s, 3H), 4.3 (s, 2H), 4.7 (s, 2H), 6.3 (s, 1H), 6.4 (bs, 2H), 6.7-6.9 (m, 3H), 7.1-7.3 (m, 3H), 10.2 (bs, 2H).

### Example 7

This example illustrates the preparation of 4-difluoromethoxy-N-[3-(2,4-difluoro-phenyl)-2-methyl-allyl]-3-methoxy-N-(1-methyl-1H-imidazol-2-ylmethyl)-benzamide (18)

Step 1: [3-(2,4-Difluoro-phenyl)-2-methyl-allyl]-(1-methyl-1H-imidazol-2-ylmethyl)-amine

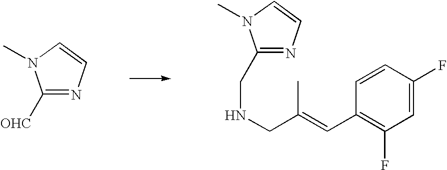

Experimental conditions analogous to described for example 1 (step3) were used with 60.6 mg (0.55 mmol) of 1-Methyl-1H-imidazole-2-carbaldehyde, 100 mg of (E)-3-(2,4-Difluoro-phenyl)-2-methyl-allylamine (0.55 mmol), 5 mL DCM, and 233 mg of sodium triacetoxyborohydride (1.1 mmol). After work-up gave 73 mg of compound used as a crude product. LC-MSD, m/z for C₁₅H₁₇F₂N₃ [M+H]+: 278.1, [M+2H]+: 279.1.

Step 2: 4-difluoromethoxy-N-[3-(2,4-difluoro-phenyl)-2-methyl-allyl]-3-methoxy-N-(1-methyl-1H-imidazol-2-ylmethyl)-benzamide

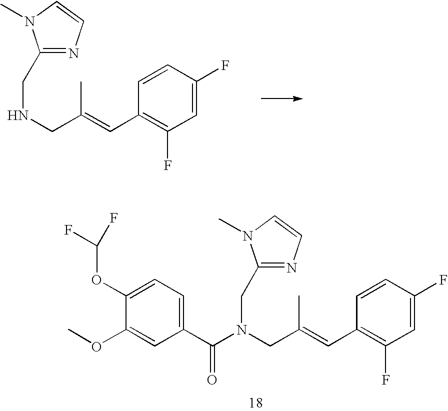

Experimental conditions analogous to described for example 1 (step 4) were used with 73 mg (0.26 mmol) of [3-(2,4-Difluoro-phenyl)-2-methyl-allyl]-(1-methyl-1H-imidazol-2-ylmethyl)-amine, 3 mL DCM, 61.5 mg (0.26 mmol) of 4-difluoromethoxy-3-methoxy-benzoyl chloride 72 μL of triethylamine (0.52 mmol.). Compound was purified using reverse phase HPLC, mobile phase with a gradient 20-80% acetonitrile. The purified TFA salt was transformed to a free base, gave 28 mg compound: Yield 18%. LC-MSD, m/z for C₂₄H₂₃F₄N₃O₃ [M+H]+: 478.2, [M+2H]+: 479.1; ¹H NMR (400 MHz, CDCl₃): δ 0.8 (m, 1H), 1.2 (s, 3H), 1.6 (s, 3H), 3.73 (s, 3H), 3.9 (s, 3H), 4.2 (s, 2H), 4.8 (s, 2H), 6.4 (s, 1H), 6.8-6.9 (m, 3H), 7.0-7.2 (m, 5H).

### Example 8

This example illustrates the preparation of N-[1-(1-Amino-cyclopentylmethyl)-pyrrolidin-2ylmethyl]-4-difluoromethoxy-N-[3-(2,4-difluoro-phenyl)-2-methyl-allyl]-3-methoxy-benzamide, (16)

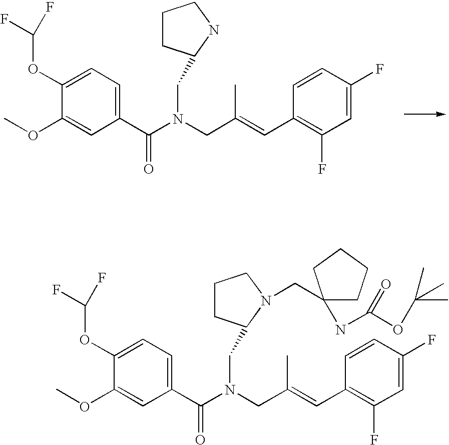

Step 1: {1-[2-({(4-Difluoromethoxy-3-methoxy-benzoyl)-[3-(2,4-difluoro-phenyl)-2-methyl-allyl]-amino}-methyl-pyrrolidin-1ylmethyl)-cyclopentyl]-carbamic acid tert-butyl ester

In 5 ml of methanol was dissolved 233 mg (0.5 mmol) 4-difluoromethoxy-N-[3-(2,4-difluoro-phenyl)-2-methyl-allyl]-3-methoxy-N-pyrrolidin-2ylmethyl-benzamide (Melikian & al WO2004058705) and 107 mg (0.5 mmol) of (1-formyl-cyclopentyl)-carbamic acid tert-butyl ester. The mixture was heated 1 h at 45° C., to this solution was added 63 mg (1.0 mmol) of sodium cyanoborohydride and continued for 1 h at 45° C., added. The reaction was not completed by LC-MS, to this mixture was added 327 mg (1.5 mmol) of (1-formyl-cyclopentyl)-carbamic acid tert-butyl ester and 189 mg (3 mmol) of sodium cyanoborohydride, and the mixture was kept at 45° C. for another 2 hours. The reaction was 95% completed, methanol was evaporated under vacuum, and the mixture was taken in ethylacetate washed with saturated sodium bicarbonate. Organic layer was dried over magnesium sulfate, filtered and concentrated under vacuum. Purification over silica gel elution with chloroform methanol 5% gave 472 mg of material. LC-MSD, m/z for C₃₅H₄₅F₄N₃O₅ [M+H]+: 664.3, [M+2H]+: 665.3; Reverse phase HPLC gradient acetonitrile 0.1% TFA 20-95% in 7 min: 4.75 min.

Step 2: N-[1-(1-Amino-cyclopentylmethyl)-pyrrolidin-2ylmethyl]-4-difluoromethoxy-N-[3-(2,4-difluoro-phenyl)-2-methyl-allyl]-3-methoxy-benzamide

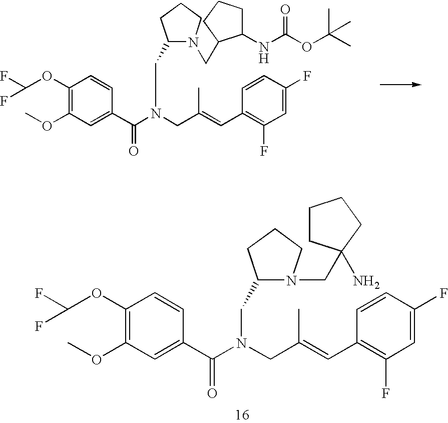

{1-[2-({(4-Difluoromethoxy-3-methoxy-benzoyl)-[3-(2,4-difluoro-phenyl)-2-methyl-allyl]-amino}-methyl-pyrrolidin-1ylmethyl)-cyclopentyl]-carbamic acid tert-butyl ester crude 0.5 mmol was dissolved in a mixture of 1.5 ml of trifluoroacetic acid and dichloromethane 5 ml, stirred at room temperature overnight. To this mixture was added saturated solution of sodium carbonate until basic pH. The mixture was extracted with dichloromethane, the combined organic layer was dried over sodium sulfate, filtered and concentrated gave 238 mg of an oil. 144 mg of this material was purified using reverse phase HPLC mobile phase acetonitrile with 0.1% TFA 20-80%. After concentrating and neutralization with sodium bicarbonate lead to 37.6 mg of compound. LC-MSD, m/z for C₃₀H₄₇F₄N₃O₃ [M+H]+: 564.3, [M+2H]+: 565.3; Reverse phase HPLC gradient acetonitrile 0.1% TFA 20-95% in 7 min: 4.26 min.

### Example 9

### 4-Difluoromethoxy-N-[3-(2,4-difluoro-phenyl)-2-methyl-allyl]-N-[ 1-(1-dimethylamino-cyclopentylmethyl)-pyrrolidin-2-ylmethyl]-3-methoxy-benzamide, (15)

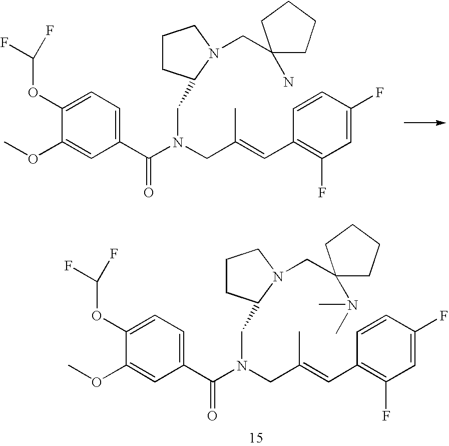

In 1.4 mL methanol at room temperature was dissolved 78 mg (0.14 mmol) of N-[1-(1-Amino-cyclopentylmethyl)-pyrrolidin-2ylmethyl]-4-difluoromethoxy-N-[3-(2,4-difluoro-phenyl)-2-methyl-allyl]-3-methoxy-benzamide, and para formaldehyde 25.2 mg (0.84 mmol). The reaction mixture is not totally homogeneous and was stirred for 1 hour, then 26 mg (0.42 mmol) of sodium cyanoborohydride was added. The reaction mixture was then stirred for overnight. The reaction was then purified by reverse phase HPLC with a mobile phase 20% to 80% acetonitrile. The compound was concentrated, yield to 40 mg of HCL salt. LC-MSD, m/z for C₃₂H₄₁F₄N₃O₃ [M+H]+: 592.3, [M+2H]+: 593.3; Reverse phase HPLC gradient acetonitrile 0.1% TFA 20-95% in 4 min: 2.28 min.

### Example 10

### 4-Difluoromethoxy-N-[3-(2,4-difluoro-phenyl)-2-methyl-allyl]-3-hydroxy-N-[1-(1-methansulfonylamino-cyclopentylmethyl)-pyrrolidin-2-ylmethyl]-benzamide (17)

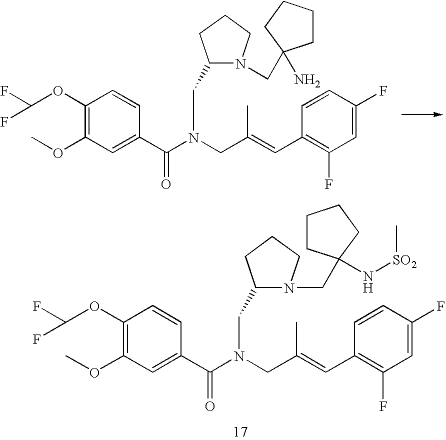

N-[1-(1-Amino-cyclopentylmethyl)-pyrrolidin-2ylmethyl]-4-difluoromethoxy-N-[3-(2,4-difluoro-phenyl)-2-methyl-allyl]-3-methoxy-benzamide 66 mg (0.117 mmol) was dissolved in 1.2 mL dichloromethane, triethylamine 65 μL (0.468 mmol) and methanesulfonic anhydride (24 mg (0.14 mmol)) was added, the mixture was stirred at room temperature. The reaction mixture passed at LC-MS showed the completion of reaction. Reaction purified using reverse phase HPLC with gradient of acetonitrile 0.1% trifluoroacetic acid 20-80% gave a compound 27.4 mg transformed to hydrochloride salt. LC-MSD, m/z for C₃₁H₃₉F₄N₃O₅S[M+H]+: 642.2, [M+2H]+: 643.2; Reverse phase HPLC gradient acetonitrile 0.1% TFA 20-95% in 4 min: 2.25 min.

### Example 11

This example illustrates the preparation of N-[1-(4-amino-cyclohexylmethyl)-pyrrolidin-2-ylmethyl]-N-[3-(2,4-difluoro-phenyl)-2-methyl-allyl]-3,4-dimethoxy-benzamide (13)

Step 1: [4-(2-{[[3-(2,4-Difluoro-phenyl)-2-methyl-allyl]-(3,4-dimethoxy-benzoyl)-amino]-methyl}-pyrrolidin-1-ylmethyl)-cyclohexyl]-carbamic acid tert-butyl ester

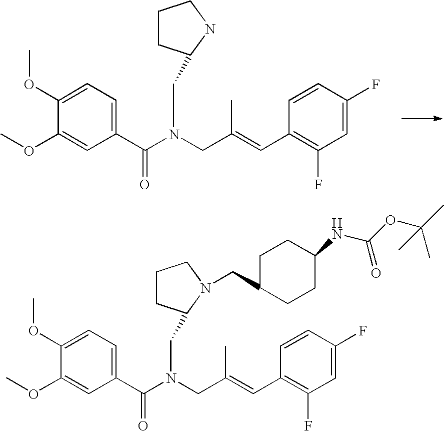

Experimental conditions analogous to described for example 1 (step 3), from 100 mg (0.214 mmol) of N-[3-(2,4-Difluoro-phenyl)-2-methyl-allyl]-3,4-dimethoxy-N-pyrrolidin-2-ylmethyl-benzamide hydrochloride (Melikian, et al, WO2004058705), 53 mg (0.23 mmol) (4-formyl-cyclohexyl)-carbamic acid tert-butyl ester, and 91 mg (0.428 mmol) of sodium triacethoxy borohydride. Purification on reverse phase HPLC acetonitrile with 0.1% TFA with a gradient 20-80% gave 72 mg of compound. LC-MSD, m/z for C₃₆H₄₉F₂N₃O₅S [M+H]+: 642.3, [M+2H]+: 643.3; Reverse phase HPLC gradient acetonitrile 0.1% TFA 20-95% in 4 min: 2.40 min.

Step 2: N-[(S)-1-(4-amino-cyclohexylmethyl)-pyrrolidin-2-ylmethyl]-N-[3-(2,4-difluoro-phenyl)-2-methyl-allyl]-3,4-dimethoxy-benzamide

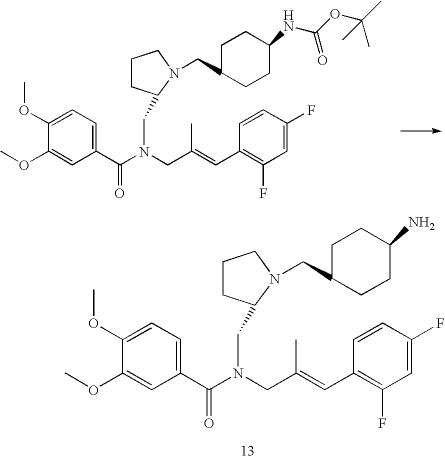

Experimental conditions analogous to described for Example 8 (Step 2), 72 mg (0.112 mmol) of [4-(2-{[[3-(2,4-Difluoro-phenyl)-2-methyl-allyl]-(3,4-dimethoxy-benzoyl)-amino]-methyl}-pyrrolidin-1-ylmethyl)-cyclohexyl]-carbamic acid tert-butyl ester was dissolved in 1 ml of dichloromethane and 200 of μL of TFA was added. The compound was neutralized with saturated bicarbonate and extracted with dichloromethane, gave 62 mg of compounds as a free base. LC-MSD, m/z for C₃₁H₄₁F₂N₃O₃ [M+H]+: 542.3, [M+2H]+: 543.3.3; Reverse phase HPLC gradient acetonitrile 0.1% TFA 20-95% in 4 min: 0.492 min.

### Example 12

### N-[(S)-1-(4-Amino-cyclohexylmethyl)-pyrrolidin-2-ylmethyl]-N-[(E)-3-(2,4-difluoro-phenyl)-2-methyl-allyl]-3,4-dimethoxy-benzamide (10)

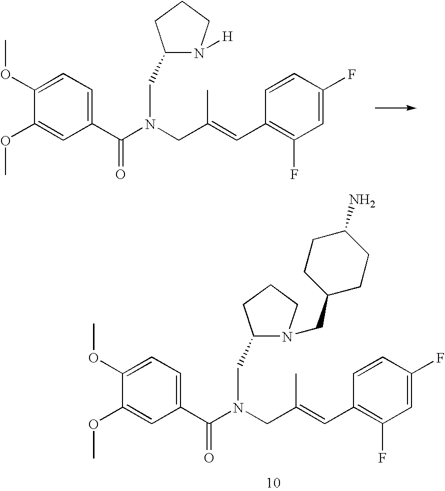

To a solution of 100 mg (0.214 mmol) of N-[(E)-3-(2,4-Difluoro-phenyl)-2-methyl-allyl]-3,4-dimethoxy-N-(S)-1-pyrrolidin-2-ylmethyl-benzamide hydrochloride in 2 mL DCM were added 49 mg (0.214 mmol) of (4-Formyl-cyclohexyl)-carbamic acid tert-butyl ester and 91 mg (0.428 mmol) of sodium triacetoxyborohydride. The mixture was stirred overnight at r.t followed by the addition of 1 mL TFA and subsequent stirring for another hour. The mixture was then evaporated and purified using reverse phase HPLC, mobile phase with a gradient 15-80% acetonitrile in 50 min. Fractions containing pure product were evaporated with 1 mL aqueous 1M HCl to yield 105 mg of the product as the dihydrochloride salt as a pale yellow oil. LC-MSD, m/z for C₃₁H₄₁F₂N₃O₃ [M+H]+: 542.3; ¹H NMR (400 MHz, CDCl₃): δ 1.1-1.3 (m, 2H), 1.5-2.4 (m, 14H), 3.0-3.4 (m, 4H), 3.8-4.3 (m, 12H), 4.6-5.0 (bs, 5H), 6.3 (s, 1H), 6.8-7.0 (m, 3H), 7.0-7.3 (m, 3H), 8.3 (bs, 3H), 10.3 (bs, 1H).

### Example 13

### N-(S)-[(E)-3-(2,4-Difluoro-phenyl)-2-methyl-allyl]-N-[(S)-1-(4-methanesulfonylamino-cyclohexylmethyl)-pyrrolidin-2-ylmethyl]-3,4-dimethoxy-benzamide (9)

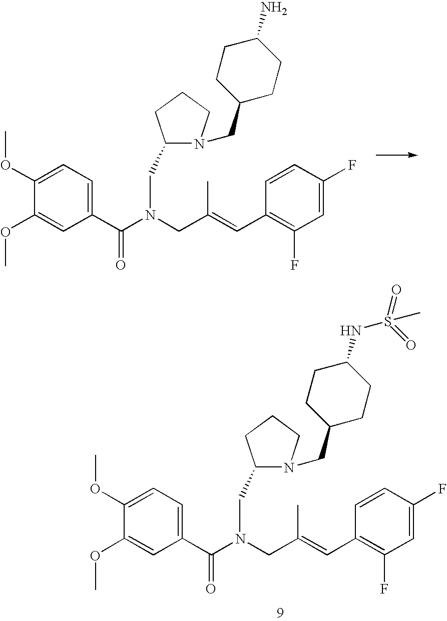

To a solution of 44 mg (0.072 mmol) of N-[(S)-1-(4-Amino-cyclohexylmethyl)-pyrrolidin-2-ylmethyl]-N-[(E)-3-(2,4-difluoro-phenyl)-2-methyl-allyl]-3,4-dimethoxy-benzamide dihydrochloride in 1 mL DCM were added 9.2 mg (0.080 mmol) of methanesulfonyl chloride and 16.2 mg (0.16 mmol) of triethylamine. The mixture was stirred for 1 hour at r.t and evaporated. It was then purified using reverse phase HPLC, mobile phase with a gradient 20-80% acetonitrile in 50 min. Fractions containing pure product were evaporated with 1 mL aqueous 1M HCl to yield 28 mg of the product as the hydrochloride salt pale yellow viscous oil. LC-MSD, m/z for C₃₂H₄₃F₂N₃O₅S [M+H]+: 620.2; ¹H NMR (400 MHz, CDCl₃): δ 1.1-1.4 (m, 4H), 1.6 (s, 3H), 1.6-2.5 (m, 10H), 2.9-3.4 (m, 3H), 3.0 (s, 3H), 3.8 (s, 3H), 3.9 (s, 3H), 3.8-4.4 (m, 7H), 6.3 (s, 1H), 6.8-6.9 (m, 3H), 7.1-7.2 (m, 2H), 7.2-7.3 (m, 1H), 11.2 (bs, 1H).

### Example 14

### 7-Methoxy-2,2-dimethyl-benzo[1,3]dioxole-5-carboxylic acid [(S)-1-(4-amino-cyclohexylmethyl)-pyrrolidin-2-ylmethyl]-((E)-2-methyl-3-phenyl-allyl)-amide (8)

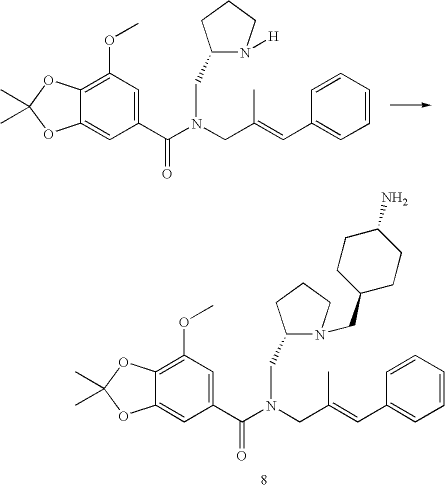

To a solution of 33 mg (0.076 mmol) of 7-Methoxy-2,2-dimethyl-benzo[1,3]dioxole-5-carboxylic acid ((E)-2-methyl-3-phenyl-allyl)-(S)-1-pyrrolidin-2-ylmethyl-amide in 1 mL DCM were added 17 mg (0.076 mmol) of (4-Formyl-cyclohexyl)-carbamic acid tert-butyl ester and 24 mg (0.113 mmol) of sodium triacetoxyborohydride. The mixture was stirred overnight at r.t followed by purification using reverse phase HPLC, mobile phase with a gradient 15-80% acetonitrile in 50 min. Fractions containing pure intermediate were evaporated, dissolved in a mixture of 1 mL DCM and 0.1 mL TFA and stirred for 2 hours at r.t. followed by neutralization with aqueous NaHCO₃ and evaporation. The mixture was then purified using reverse phase HPLC, mobile phase with a gradient 15-80% acetonitrile in 50 min. Fractions containing pure product were evaporated, the residue was dissolved in DCM, which was washed with aqueous NaHCO₃, dried with anhydrous MgSO₄ and evaporated in vacuo to yield 17 mg of the product as the free base pale yellow viscous oil. LC-MSD, m/z for C₃₃H₄₅N₃O₄ [M+H]+: 548.3; ¹H NMR (400 MHz, CDCl₃): δ 0.8-3.6 (m, 32H), 3.9 (s, 3H), 4.1-4.4 (m, 2H), 6.4 (s, 1H), 6.5 (s, 1H), 6.6 (s, 1H), 7.2-7.4 (m, 5H).

### Example 15

### Methoxy-2,2-dimethyl-benzo[1,3]dioxole-5-carboxylic acid [(S)-1-(4-isopropylamino-cyclohexylmethyl)-pyrrolidin-2-ylmethyl]-((E)-2-methyl-3-phenyl-allyl)-amide (6)

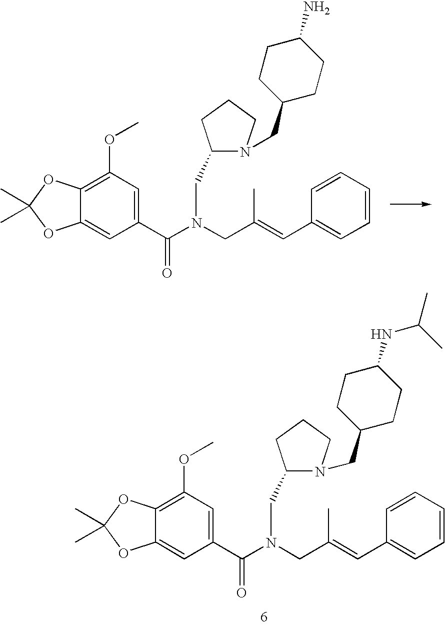

12 mg (0.022 mmol) of 7-Methoxy-2,2-dimethyl-benzo[1,3]dioxole-5-carboxylic acid [(S)-1-(4-amino-cyclohexylmethyl)-pyrrolidin-2-ylmethyl]-((E)-2-methyl-3-phenyl-allyl)-amide were dissolved in a mixture of 0.9 mL DCM and 0.1 mL acetone. To this solution 9.2 mg (0.044 mmol) of sodium triacetoxyborohydride were added and the mixture was stirred at r.t. overnight. The mixture was then evaporated and purified using reverse phase HPLC, mobile phase with a gradient 15-80% acetonitrile in 50 min. Fractions containing pure product were evaporated, the residue was dissolved in DCM, which was washed with aqueous sodium bicarbonate, dried with anhydrous MgSO₄ and evaporated in vacuum to yield 6.5 mg of the product as the free base pale yellow viscous oil. LC-MSD, m/z for C₃₆H₅₁N₃O₄ [M-methylcinnamyl]+: 460.3 [M+H]+: 590.4; ¹H NMR (400 MHz, CDCl₃): δ 1.1 (dd, 6H), 1.2-3.7 (m, 31H), 3.8 (s, 3H), 4.1-4.3 (m, 2H), 6.4 (s, 1H), 6.5 (s, 1H), 6.6 (s, 1H), 7.2-7.4 (m, 5H).

### Example 16

### N-[(S)-1-((1S,3R)-3-Amino-cyclopentylmethyl)-pyrrolidin-2-ylmethyl]-N-[(E)-3-(2,4-difluoro-phenyl)-2-methyl-allyl]-3,4-dimethoxy-benzamide (7)

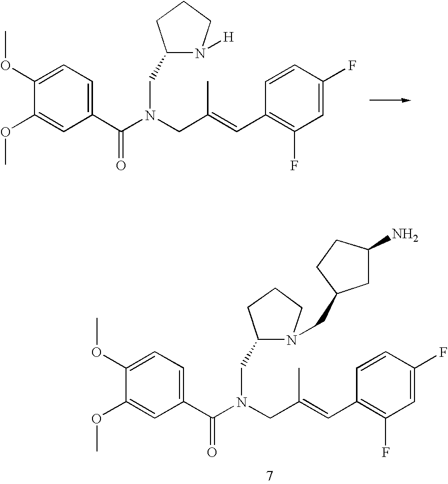

A procedure analogous to Example 14 was used with 100 mg (0.214 mmol) of N-[(E)-3-(2,4-difluoro-phenyl)-2-methyl-allyl]-3,4-dimethoxy-N-(S)-1-pyrrolidin-2-ylmethyl-benzamide hydrochloride in 2 mL DCM, 91 mg (0.428 mmol) of sodium triacetoxyborohydride and 49 mg (0.214 mmol) of ((1R,3S)-3-Formyl-cyclopentyl)-carbamic acid tert-butyl ester. The mixture was then evaporated and purified using reverse phase HPLC, mobile phase with a gradient 25-60% acetonitrile in 50 min. Fractions containing pure product were evaporated with 2 mL aqueous 1M HCl to yield 140 mg of the product as the dihydrochloride salt as a pale yellow viscous oil. LC-MSD, m/z for C₃₀H₃₉F₂N₃O₃ [M+H]+: 528.3; ¹H NMR (400 MHz, CDCl₃): δ 1.6 (s, 3H), 1.7-1.9 (m, 2H), 1.9-2.2 (m, 6H), 2.3-2.4 (m, 1H), 2.4-2.6 (m, 2H), 3.0 (bs, 8H), 3.1-3.3 (m, 2H), 3.6-4.1 (m, 4H), 3.85 (s, 3H), 3.9 (s, 3H), 4.1-4.3 (m, 2H), 6.3 (s, 1H), 6.7-6.9 (m, 3H), 7.0-7.2 (m, 2H), 7.2-7.3 (m, 1H), 8.5 (bs, 3H), 10.6 (bs, 1H).

### Example 17

### N-[(E)-3-(2,4-Difluoro-phenyl)-2-methyl-allyl]-N-[(S)-1-((1 S,3R)-3-isopropylamino-cyclopentylmethyl)-pyrrolidin-2-ylmethyl]-3,4-dimethoxy-benzamide

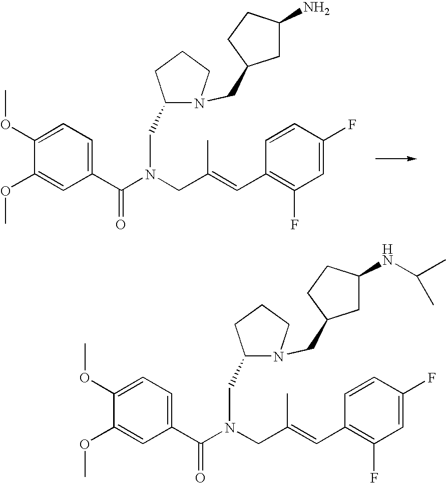

A procedure analogous to example 15 was used with 24 mg (0.040 mmol) of N-[(S)-1-((1S,3R)-3-Amino-cyclopentylmethyl)-pyrrolidin-2-ylmethyl]-N-[(E)-3-(2,4-difluoro-phenyl)-2-methyl-allyl]-3,4-dimethoxy-benzamide dihydrochloride, 1.8 mL DCM, 0.2 mL acetone and 25 mg (0.12 mmol) of sodium triacetoxyborohydride. The mixture was then evaporated and purified using reverse phase HPLC, mobile phase with a gradient 15-80% acetonitrile in 50 min. Fractions containing pure product were evaporated with 1 mL aqueous 1M HCl to yield 25 mg of the product as the dihydrochloride salt as a pale yellow viscous oil. LC-MSD, m/z for C₃₃H₄₅F₂N₃O₃ [M+2H]++: 285.6 [M+H]+: 570.3; ¹H NMR (400 MHz, CDCl₃): δ 1.4 (dd, 6H), 1.6 (s, 3H), 1.8-2.4 (m, 7H), 2.5-2.6 (m, 2H), 3.1-3.3 (m, 3H), 3.6-4.0 (m, 14H), 4.1-4.3 (m, 3H), 6.3 (s, 1H), 6.7-6.9 (m, 3H), 7.1-7.3 (m, 4H), 9.1 (bs, 1H), 9.2 (bs, 1H), 10.6 (bs, 1H).

### Example 18

To demonstrate that the compounds described above are useful modulators for chemokine binding to CCXCKR2, the compounds were screened in vitro to determine their ability to displace SDF-1 from the CCXCKR2 receptor at multiple concentrations. The compounds were combined with mammary gland cells expressing the CCXCKR2 receptor in the presence of the ¹²⁵I-labeled chemokine as detailed in Determination of IC₅₀ values, Reagents and Cells (see below). The ability of the compounds to displace the labeled chemokine from the CCXCKR2 receptor sites at multiple concentrations was then determined with the screening process.

Compounds that were deemed effective modulators were able to displace at least 50% of the SDF-1 from the CCXCKR2 receptor at concentrations at or below 2.1 micromolar (μM), as represented by the notation (+) for certain exemplary compounds shown in Table B below, and more preferably at concentrations at or below 700 nanomolar (nM) (++). At present, especially preferred compounds can displace at least 50% of the SDF-1 from the CCXCKR2 receptor at concentrations at or below 500 nM (+++). As stated above, Exemplary compounds that met these criteria are reproduced in Table B below. All compounds were prepared as described in the Examples above, or by related methods substituting readily available starting materials.

TABLE B

| | | Ac- |
|---|---|---|
| No | Structure | tivity |
| 1 | 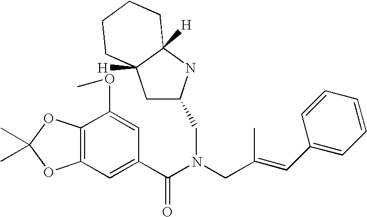 | +++ |
| 2 | 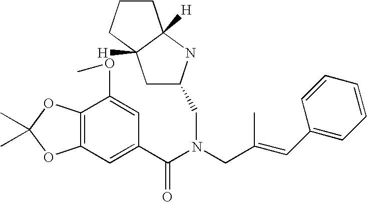 | +++ |
| 3 | 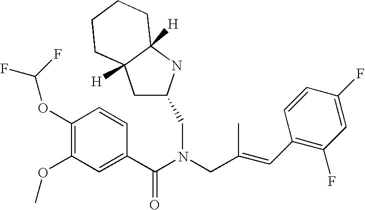 | +++ |
| 4 | 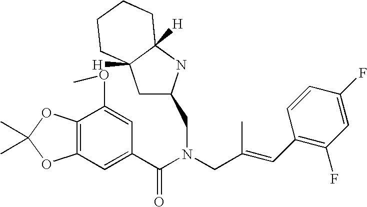 | +++ |
| 5 | 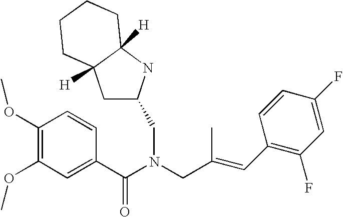 | +++ |
| 6 | 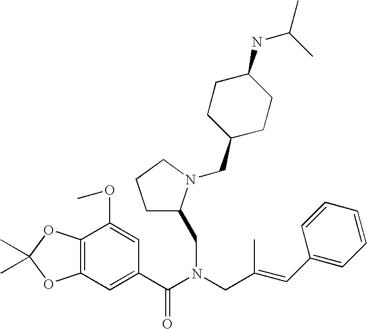 | +++ |
| 7 | 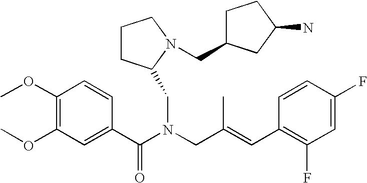 | +++ |
| 8 | 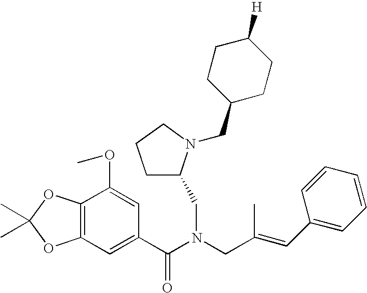 | +++ |
| 9 | 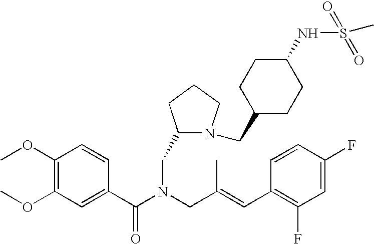 | +++ |
| 10 | 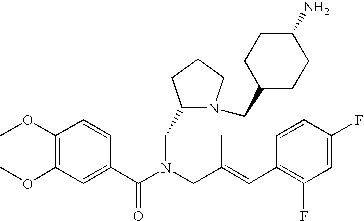 | +++ |
| 11 | 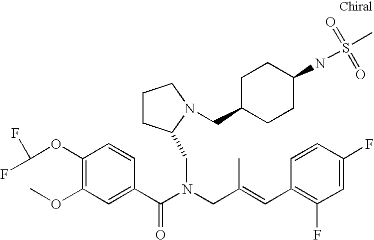 | +++ |
| 13 | 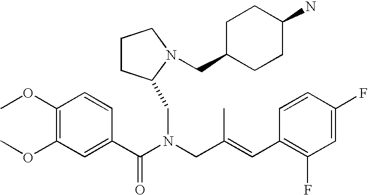 | +++ |
| 14 | 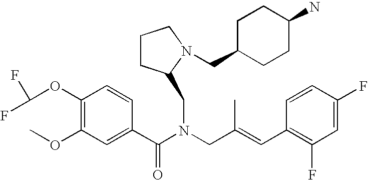 | +++ |
| 15 | 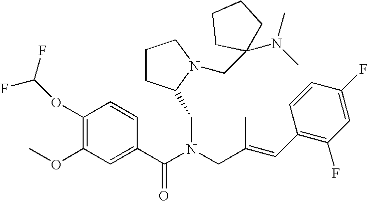 | +++ |
| 16 | 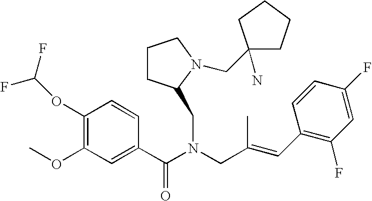 | ++ |
| 17 | 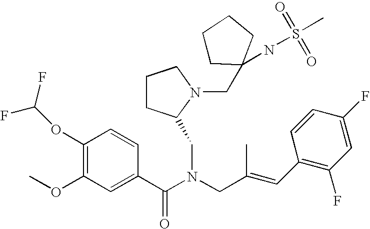 | +++ |
| 18 | 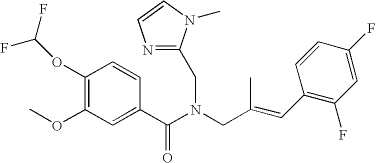 | + |
| 19 | 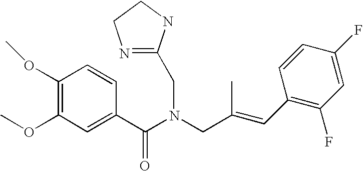 | +++ |
| 20 | 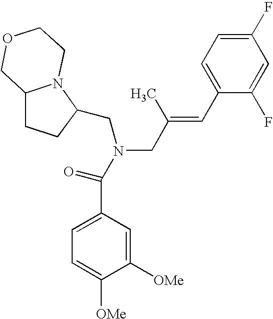 | + |

1. Determination Of IC₅₀ Values.

Reagents and Cells. ¹²⁵I-labeled SDF-1 was purchased from Perkin-Elmer Life Sciences, Inc. (Boston, Mass.). The MCF-7 (adenocarcinoma; mammary gland) cell line was obtained from the American Type Culture Collection (Manassas, Va.) or and was cultured in DMEM (Mediatech, Herndon, Va.) supplemented with 10% fetal bovine serum (FBS) (HyClone Logan, Utah) and bovine insulin (0.01 mg/mL) (Sigma, St. Louis, Mo.) at 37° C. in a humidified incubator at a 5% CO₂/air mixture. CCXCKR2 transfected MDA-MB-435S were produced as described below. MDA-MB-435S human breast cancer line, was purchased from ATCC, and cultured in DMEM/10% FBS medium. The complete coding sequence of the gene encoding CCXCKR2 (a.k.a.CXCR⁷, hRDC1), was isolated from MCF-7 cells using μMACs mRNA isolation kit (Miltenyi Biotec, Auburn, Calif.). DNA contamination was removed by DNase digestion via RNeasy columns (Qiagen, Inc., Valencia, Calif.) and cDNA was generated using GeneAmp RNA PCR Core Kit (Applied Biosystems, Foster City, Calif.). PCR of cDNA samples was performed using Taq PCR Master Mix kit (Qiagen, Inc.) and hRDC1 primers harboring 5′ and 3′ Not I sites

(hRDC1F 5′ GAATGCGGCCGCTATGGATCTGCATCTCTTCGACT-3′,
hRDC1R 5′-GAATGCGGCCGCTCATTTGGTGCTCTGCTCCAAG-3′)

Not I digested PCR
product was ligated into Not I digested pcDNA3.1(+)(Invitrogen, Carlsbad, Calif.) and screened for orientation and sequence confirmed. Plasmid DNA was then isolated from overnight bacterial cultures by Maxiprep (Qiagen, Inc.). Plasmid DNA (10 μg) was added to MDA-MB-435s cells and cells were electroporated (0.22 kV, 960 uF) via Gene Pulser (Biorad laboratories, Hercules, Calif.). 48 hr post-electroporation, cells were transferred to selection medium (1000 ug/ml G418).

Binding Analysis. Target compounds were tested to determine their ability to bind with CCXCKR2 sites on MCF-7 and/or MDA-MB-435S cells. Efficiency-maximized radioligand binding using filtration protocols as described in Dairaghi D J, et al., HHV8-encoded vMIP-I selectively engages chemokine receptor CCR5. Agonist and antagonist profiles of viral chemokines., J. Biol. Chem. 1999 Jul. 30; 274(31): 21569-74 and Gosling J, et al., Cutting edge: identification of a novel chemokine receptor that binds dendritic cell- and T cell-active chemokines including ELC, SLC, and TECK., J. Immunol. 2000 Mar. 15; 164(6):2851-6 was used.

In these assays, MCF-7 and/or MDA-MB-435S cells were interrogated with the target compounds and the ability of these compounds to displace ¹²⁵I radiolabeled SDF-1 was assessed using the protocol described in Dairaghi and Gosling. The target compounds were added to the plate to the indicated concentration and were then incubated with cells followed by the addition of radiolabeled chemokine (¹²⁵I SDF-1) for 3 hr at 4° C. in the following binding medium (25 mM HEPES, 140 mM NaCl, 1 mM CaCl₂, 5 mM MgCl₂ and 0.2% bovine serum albumin, adjusted to pH 7.1). All assays were then incubated for 3 hrs at 4° C. with gentle agitation. Following incubation in all binding assays, reactions were aspirated onto PEI-treated GF/B glass filters (Packard) using a cell harvester (Packard) and washed twice (25 mM HEPES, 500 mM NaCl, 1 mM CaCl₂, 5 mM MgCl₂, adjusted to pH 7.1). Scintillant (MicroScint 10, Packard) was added to the wells, and the filters were counted in a Packard Topcount scintillation counter. Data were analyzed and plotted using Prism (GraphPad Prism version 3.0a for Macintosh, GraphPad Software, www.graphpad.com).

One of ordinary skill in the art will recognize from the provided description, figures, and examples, that modifications and changes can be made to the various embodiments of the invention without departing from the scope of the invention defined by the following claims and their equivalents.

SEQUENCE LISTING

SEQ ID NO:1 CCXCKR2 coding sequence
ATGGATCTGCATCTCTTCGACTACTCAGAGCCAGGGAACTTCTCGGACAT
CAGCTGGCCATGCAACAGCAGCGACTGCATCGTGGTGGACACGGTGATGT
GTCCCAACATGCCCAACAAAAGCGTCCTGCTCTACACGCTCTCCTTCATT
TACATTTTCATCTTCGTCATCGGCATGATTGCCAACTCCGTGGTGGTCTG
GGTGAATATCCAGGCCAAGACCACAGGCTATGACACGCACTGCTACATCT
TGAACCTGGCCATTGCCGACCTGTGGGTTGTCCTCACCATCCCAGTCTGG
GTGGTCAGTCTCGTGCAGCACAACCAGTGGCCCATGGGCGAGCTCACGTG
CAAAGTCACACACCTCATCTTCTCCATCAACCTCTTCGGCAGCATTTTCT
TCCTCACGTGCATGAGCGTGGACCGCTACCTCTCCATCACCTACTTCACC
AACACCCCCAGCAGCAGGAAGAAGATGGTACGCCGTGTCGTCTGCATCCT
GGTGTGGCTGCTGGCCTTCTGCGTGTCTCTGCCTGACACCTACTACCTGA
AGACCGTCACGTCTGCGTCCAACAATGAGACCTACTGCCGGTCCTTCTAC
CCCGAGCACAGCATCAAGGAGTGGCTGATCGGCATGGAGCTGGTCTCCGT
TGTCTTGGGCTTTGCCGTTCCCTTCTCCATTATCGCTGTCTTCTACTTCC
TGCTGGCCAGAGCCATCTCGGCGTCCAGTGACCAGGAGAAGCACAGCAGC
CGGAAGATCATCTTCTCCTACGTGGTGGTCTTCCTTGTCTGCTGGCTGCC
CTACCACGTGGCGGTGCTGCTGGACATCTTCTCCATCCTGCACTACATCC
CTTTCACCTGCCGGCTGGAGCACGCCCTCTTCACGGCCCTGCATGTCACA
CAGTGCCTGTCGCTGGTGCACTGCTGCGTCAACCCTGTCCTCTACAGCTT
CATCAATCGCAACTACAGGTACGAGCTGATGAAGGCCTTCATCTTCAAGT
ACTCGGCCAAAACAGGGCTCACCAAGCTCATCGATGCCTCCAGAGTCTCA
GAGACGGAGTACTCTGCCTTGGAGCAGAGCACCAAATGA
SEQ ID NO:2 CCXCKR2 amino acid sequence
MDLHLFDYSEPGNFSDISWPCNSSDCIVVDTVMCPNMPNKSVLLYTLSFI
YIFIFVIGMIANSVVVWVNIQAKTTGYDTHCYILNLAIADLWVVLTIPVW
VVSLVQHNQWPMGELTCKVTHLIFSINLFGSIFFLTCMSVDRYLSITYFT
NTPSSRKKMVRRVVCILVWLLAFCVSLPDTYYLKTVTSASNNETYCRSFY
PEHSIKEWLIGMELVSVVLGFAVPFSIIAVFYFLLARAISASSDQEKHSS
RKIIFSYVVVFLVCWLPYHVAVLLDIFSILHYIPFTCRLEHALFTALHVT
QCLSLVHCCVNPVLYSFINRNYRYELMKAFIFKYSAKTGLTKLIDASRVS
ETEYSALEQSTK
SEQ ID NO:3 CCXCKR2.2 coding sequence
ATGGATCTGCACCTCTTCGACTACGCCGAGCCAGGCAACTTCTCGGACAT
CAGCTGGCCATGCAACAGCAGCGACTGCATCGTGGTGGACACGGTGATGT
GTCCCAACATGCCCAACAAAAGCGTCCTGCTCTACACGCTCTCCTTCATT
TACATTTTCATCTTCGTCATCGGCATGATTGCCAACTCCGTGGTGGTCTG
GGTGAATATCCAGGCCAAGACCACAGGCTATGACACGCACTGCTACATCT
TGAACCTGGCCATTGCCGACCTGTGGGTTGTCCTCACCATCCCAGTCTGG
GTGGTCAGTCTCGTGCAGCACAACCAGTGGCCCATGGGCGAGCTCACGTG
CAAAGTCACACACCTCATCTTCTCCATCAACCTCTTCAGCGGCATTTTCT
TCCTCACGTGCATGAGCGTGGACCGCTACCTCTCCATCACCTACTTCACC
AACACCCCCAGCAGCAGGAAGAAGATGGTACGCCGTGTCGTCTGCATCCT
GGTGTGGCTGCTGGCCTTCTGCGTGTCTCTGCCTGACACCTACTACCTGA
AGACCGTCACGTCTGCGTCCAACAATGAGACCTACTGCCGGTCCTTCTAC
CCCGAGCACAGCATCAAGGAGTGGCTGATCGGCATGGAGCTGGTCTCCGT
TGTCTTGGGCTTTGCCGTTCCCTTCTCCATTATCGCTGTCTTCTACTTCC
TGCTGGCCAGAGCCATCTCGGCGTCCAGTGACCAGGAGAAGCACAGCAGC
CGGAAGATCATCTTCTCCTACGTGGTGGTCTTCCTTGTCTGCTGGCTGCC
CTACCACGTGGCGGTGCTGCTGGACATCTTCTCCATCCTGCACTACATCC
CTTTCACCTGCCGGCTGGAGCACGCCCTCTTCACGGCCCTGCATGTCACA
CAGTGCCTGTCGCTGGTGCACTGCTGCGTCAACCCTGTCCTCTACAGCTT
CATCAATCGCAACTACAGGTACGAGCTGATGAAGGCCTTCATCTTCAAGT
ACTCGGCCAAAACAGGGCTCACCAAGCTCATCGATGCCTCCAGAGTGTCG
GAGACGGAGTACTCCGCCTTGGAGCAAAACGCCAAGTGA
SEQ ID NO:4 CCXCKR2.2 amino acid sequence
MDLHLFDYAEPGNFSDISWPCNSSDCIVVDTVMCPNMPNKSVLLYTLSFI
YIFIFVIGMIANSVVVWVNIQAKTTGYDTHCYILNLAIADLWVVLTIPVW
VVSLVQHNQWPMGELTCKVTHLIFSINLFSGIFFLTCMSVDRYLSITYFT
NTPSSRKKMVRRVVCILVWLLAFCVSLPDTYYLKTVTSASNNETYCRSFY
PEHSIKEWLIGMELVSVVLGFAVPFSIIAVFYFLLARAISASSDQEKHSS
RKIIFSYVVVFLVCWLPYHVAVLLDIFSILHYIPFTCRLEHALFTALHVT
QCLSLVHCCVNPVLYSFINRNYRYELMKAFIFKYSAKTGLTKLIDASRVS
ETEYSALEQNAK
SEQ ID NO:5 CCXCKR2.3 coding sequence
ATGGATCTGCATCTCTTCGACTACTCAGAGCCAGGGAACTTCTCGGACAT
CAGCTGGCCATGCAACAGCAGCGACTGCATCGTGGTGGACACGGTGATGT
GTCCCAACATGCCCAACAAAAGCGTCCTGCTCTACACGCTCTCCTTCATT
TACATTTTCATCTTCGTCATCGGCATGATTGCCAACTCCGTGGTGGTCTG
GGTGAATATCCAGGCCAAGACCACAGGCTATGACACGCACTGCTACATCT
TGAACCTGGCCATTGCCGACCTGTGGGTTGTCCTCACCATCCCAGTCTGG
GTGGTCAGTCTCGTGCAGCACAACCAGTGGCCCATGGGCGAGCTCACGTG
CAAAGTCACACACCTCATCTTCTCCATCAACCTCTTCGGCAGCATTTTCT
TCCTCACGTGCATGAGCGTGGACCGCTACCTCTCCATCACCTACTTCACC
AACACCCCCAGCAGCAGGAAGAAGATGGTACGCCGTGTCGTCTGCATCCT
GGTGTGGCTGCTGGCCTTCTGCGTGTCTCTGCCTGACACCTACTACCTGA
AGACCGTCACGTCTGCGTCCAACAATGAGACCTACTGCCGGTCCTTCTAC
CCCGAGCACAGCATCAAGGAGTGGCTGATCGGCATGGAGCTGGTCTCCGT
TGTCTTGGGCTTTGCCGTTCCCTTCTCCATTGTCGCTGTCTTCTACTTCC
TGCTGGCCAGAGCCATCTCGGCGTCCAGTGACCAGGAGAAGCACAGCAGC
CGGAAGATCATCTTCTCCTACGTGGTGGTCTTCCTTGTCTGCTGGTTGCC
CTACCACGTGGCGGTGCTGCTGGACATCTTCTCCATCCTGCACTACATCC
CTTTCACCTGCCGGCTGGAGCACGCCCTCTTCACGGCCCTGCATGTCACA
CAGTGCCTGTCGCTGGTGCACTGCTGCGTCAACCCTGTCCTCTACAGCTT
CATCAATCGCAACTACAGGTACGAGCTGATGAAGGCCTTCATCTTCAAGT
ACTCGGCCAAAACAGGGCTCACCAAGCTCATCGATGCCTCCAGAGTCTCA
GAGACGGAGTACTCTGCCTTGGAGCAGAGCACCAAATGA
SEQ ID NO:6 CCXCRR2.3 amino acid sequence
MDLHLFDYSEPGNFSDISWPCNSSDCIVVDTVMCPNMPNKSVLLYTLSFI
YIFIFVIGMIANSVVVWVNIQAKTTGYDTHCYILNLAIADLWVVLTIPVW
VVSLVQHNQWPMGELTCKVTHLIFSINLFGSIFFLTCMSVDRYLSITYFT
NTPSSRKKMVRRVVCILVWLLAFCVSLPDTYYLKTVTSASNNETYCRSFY
PEHSIKEWLIGMELVSVVLGFAVPFSIVAVFYFLLARAISASSDQEKHSS
RKIIFSYVVVFLVCWLPYHVAVLLDIFSILHYIPFTCRLEHALFTALHVT
QCLSLVHCCVNPVLYSFINRNYRYELMKAFIFKYSAKTGLTKLIDASRVS
ETEYSALEQSTK
SEQ ID NO:7 CCXCKR2.4 coding sequence
ATGGATCTGCATCTCTTCGACTACTCAGAGCCAGGGAACTTCTCGGACAT
CAGCTGGCCATGCAACAGCAGCGACTGCATCGTGGTGGACACGGTGATGT
GTCCCAACATGCCCAACAAAAGCGTCCTGCTCTACACGCTCTCCTTCATT
TACATTTTCATCTTCGTCATCGGCATGATTGCCAACTCCGTGGTGGTCTG
GGTGAATATCCAGGCCAAGACCACAGGCTATGACACGCACTGCTACATCT
TGAACCTGGCCATTGCCGACCTGTGGGTTGTCCTCACCATCCCAGTCTGG
GTGGTCAGTCTCGTGCAGCACAACCAGTGGCCCATGGGCGAGCTCACGTG
CAAAGTCACACACCTCATCTTCTCCATCAACCTCTTCGGCAGCATTTTCT
TCCTCACGTGCATGAGCGTGGACCGCTACCTCTCCATCACCTACTTCACC
AACACCCCCAGCAGCAGGAAGAAGATGGTACGCCGTGTCGTCTGCATCCT
GGTGTGGCTGCTGGCCTTCTGCGTGTCTCTGCCTGACACCTACTACCTGA
AGACCGTCACGTCTGCGTCCAACAATGAGACCTACTGCCGGTCCTTCTAC
CCCGAGCACAGCATCAAGGAGTGGCTGATCGGCATGGAGCTGGTCTCCGT
TGTCTTGGGCTTTGCCGTTCCCTTCTCCATTATCGCTGTCTTCTACTTCC
TGCTGGCCAGAGCCATCTCGGCGTCCAGTGACCAGGAGAAGCACAGCAGC
CGGAAGATCATCTTCTCCTACGTGGTGGTCTTCCTTGTCTGCTGGCTGCC
CTACCACGTGGCGGTGCTGCTGGACATCTTCTCCATCCTGCACTACATCC
CTTTCACCTGCCGGCTGGAGCACGCCCTCTTCACGGCCCTGCATGTCACA
CAGTGCCTGTCGCTGGTGCACTGCTGCGTCAACCCTGTCCTCTACAGCTT
CATCAATCGCAACTACAGGTACGAGCTGATGAAGGCCTTCATCTTCAAGT
ACTCGGCCAAAACAGGGCTCACCAAGCTCATCGATGCCTCCAGAGTCTCA
GAGACGGAGTACTCTGCCTTGGAGCAGAGCACCAAATGA
SEQ ID NO:8 CCXCKR2.4 amino acid sequence
MDLHLFDYSEPGNFSDISWPCNSSDCIVVDTVMCPNMPNKSVLLYTLSFI
YIFIFVIGMIANSVVVWVNIQAKTTGYDTHCYILNLAIADLWVVLTIPVW
VVSLVQHNQWPMGELTCKVTHLIFSINLFGSIFFLTCMSVDRYLSITYFT
NTPSSRKKMVRRVVCILVWLLAFCVSLPDTYYLKTVTSASNNETYCRSFY
PEHSIKEWLIGMELVSVVLGFAVPFSILAVFYFLLARAISASSDQEKHSS
RKIIFSYVVVFLVCWLPYHVAVLLDIFSILHYIPFTCRLEHALFTALHVT
QCLSLVHCCVNPVLYSFINRNYRYELMKAFIFKYSAKTGLTKLIDASRVS
ETEYSALEQSTK
SEQ ID NO:9 CCXCKR2.5 coding sequence
ATGGATCTGCATCTCTTCGACTACTCAGAGCCAGGGAACTTCTCGGACAT
CAGCTGGCCGTGCAACAGCAGCGACTGCATCGTGGTGGACACGGTGATGT
GTCCCAACATGCCCAACAAAAGCGTCCTGCTCTACACGCTCTCCTTCATT
TACATTTTCATCTTCGTCATCGGCATGATTGCCAACTCCGTGGTGGTCTG
GGTGAATATCCAGGCCAAGACCACAGGCTATGACACGCACTGCTACATCT
TGAACCTGGCCATTGCCGACCTGTGGGTTGTCCTCACCATCCCAGTCTGG
GTGGTCAGTCTCGTGCAGCACAACCAGTGGCCCATGGGCGAGCTCACGTG
CAAAGTCACACACCTCATCTTCTCCATCAACCTCTTCAGCAGCATTTTCT
TCCTCACGTGCATGAGCGTGGACCGCTACCTCTCCATCACCTACTTCACC
AACACCCCCAGCAGCAGGAAGAAGATGGTACGCCGTGTCGTCTGCATCCT
GGTGTGGCTGCTGGCCTTCTGCGTGTCTCTGCCTGACACCTACTACCTGA
AGACCGTCACGTCTGCGTCCAACAATGAGACCTACTGCCGGTCCTTCTAC
CCCGAGCACAGCATCAAGGAGTGGCTGATCGGCATGGAGCTGGTCTCCGT
TGTCTTGGGCTTTGCCGTTCCCTTCTCCATTATCGCTGTCTTCTACTTCC
TGCTGGCCAGAGCCATCTCGGCGTCCAGTGACCAGGAGAAGCACAGCAGC
CGGAAGATCATCTTCTCCTACGTGGTGGTCTTCCTTGTCTGCTGGTTGCC
CTACCACGTGGCGGTGCTGCTGGACATCTTCTCCATCCTGCACTACATCC
CTTTCACCTGCCGGCTGGAGCACGCCCTCTTCACGGCCCTGCATGTCACA
CAGTGCCTGTCGCTGGTGCACTGCTGCGTCAACCCTGTCCTCTACAGCTT
CATCAATCGCAACTACAGGTACGAGCTGATGAAGGCCTTCATCTTCAAGT
ACTCGGCCAAAACAGGGCTCACCAAGCTCATCGATGCCTCCAGAGTCTCA
GAGACGGAGTACTCCGCCTTGGAGCAGAGCACCAAATGA
SEQ ID NO:10 CCXCKR2.5 amino acid sequence
MDLHLFDYSEPGNFSDISWPCNSSDCIVVDTVMCPNMPNKSVLLYTLSFI
YIFIFVIGMIANSVVVWVNIQAKTTGYDTHCYILNLAIADLWVVLTIPVW
VVSLVQHNQWPMGELTCKVTHLIFSINLFSSIFFLTCMSVDRYLSITYFT
NTPSSRKKMVRRVVCILVWLLAFCVSLPDTYYLKTVTSASNNETYCRSFY
PEHSIKEWLIGMELVSVVLGFAVPFSIIAVFYFLLARAISASSDQEKHSS
RKIIFSYVVVFLVCWLPYHVAVLLDIFSILHYIPFTCRLEHALFTALHVT
QCLSLVHCCVNPVLYSFINRNYRYELMKAFIFKYSAKTGLTKLIDASRVS
ETEYSALEQSTK

1. A compound having a formula selected from the group consisting of: 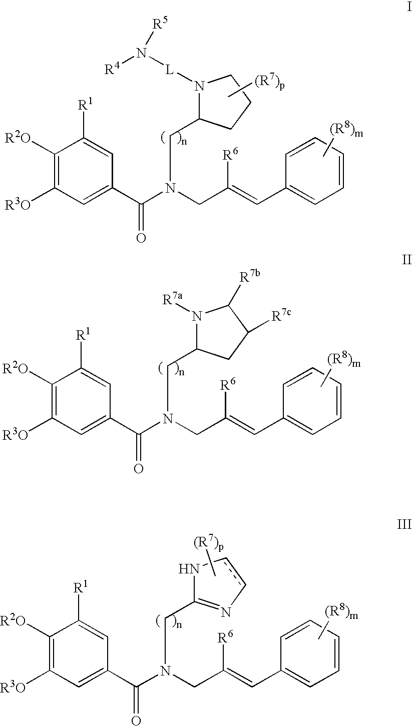
and all pharmaceutically acceptable salts and hydrates thereof, wherein
the subscript m is an integer of from 0 to 3;
the subscript n is an integer of from 1 to 3;
the subscript p is an integer of from 0 to 3;
the dotted line of formula III indicates the presence of an optional double bond;
L is a C₁₋₄ alkylC₃₋₆ cycloalkyl linking group;
R¹ is a member selected from the group consisting of hydrogen, halogen, C₁₋₈ alkoxy, C₁₋₈ alkyl, C₁₋₈ haloalkyl, C₃₋₆ cycloalkyl, C₃₋₆ cycloalkoxy, C₃₋₆ cycloalkyl, C₁₋₄ alkyl and C₃₋₆ cycloalkyl C₁₋₄ alkoxy;
R² and R³ are each members independently selected from C₁₋₈ alkyl and C₁₋₈ haloalkyl, or are optionally combined with the oxygen atoms to which each is attached to from a five- to ten-membered ring;
R⁴ and R⁵ are each independently selected from the group consisting of H, C₁₋₈ alkyl, C₁₋₈ haloalkyl, C₃₋₆ cycloalkyl, —COR^{a}, —CO₂R^{a}, —CONR^{a}R^{b}, —SO₂R^{a} and —SO₂NR^{a}R^{b};
R⁶ is selected from the group consisting of H and C₁₋₈ alkyl;
each R⁷ substituent is independently selected from the group consisting of C₁₋₈ alkyl, C₁₋₈ haloalkyl, C₃₋₆ cycloalkyl, C₂₋₈ alkenyl, C₂₋₈ alkynyl, —OR^{a}, —NR^{a}R^{b}, —COR^{a}, —CO₂R^{a}, —CONR^{a}R^{b}, —NR^{a}COR^{b}, —SO₂R^{a}, —X¹COR^{a}, —X¹CO₂R^{a}, —X¹CONR^{a}R^{b}, —X¹NR^{a}COR^{b}, —X¹SO₂R^{a}, —X¹SO₂NR^{a}R^{b}, —X¹NR^{a}R^{b} and —X¹OR^{a};
wherein each X¹ is a member selected from the group consisting of C₁₋₄ alkylene and C₂₋₄ alkenylene and each R^{a} and R^{b} is independently selected from the group consisting of hydrogen, C₁₋₈ alkyl, C₁₋₈ haloalkyl, C₃₋₆ cycloalkyl and aryl-C₁₋₄ alkyl,
two adjacent members of R^{7a}, R^{7b} and R^{7c} are combined to form a fused five or six-membered ring that is carbocyclic or heterocyclic and optionally substituted with from one to three substituents; and the remaining member of R^{7a} and R^{7c} is R⁷;
each R⁸ is independently selected from the group consisting of halogen, C₁₋₈ alkyl, C₁₋₈ haloalkyl, C₃₋₆ cycloalkyl, C₂₋₈ alkenyl, C₂₋₈ alkynyl, —OR^{a}, —NR^{a}R^{b}, —COR^{a}, —CO₂R^{a}, —CONR^{a}R^{b}, —NR^{a}COR^{b}, —SO₂R^{a}, —X¹COR^{a}, —X¹CO₂R^{a}, —X¹CONR^{a}R^{b}, —X¹NR^{a}COR^{b}, —X¹SO₂R^{a}, —X¹SO₂NR^{a}R^{b}, —X¹NR^{a}R^{b} and —X¹OR^{a};
and wherein the aliphatic portions of each of said R⁷ substituents and the ring formed by combining R^{7a} with R^{7b} or by combining R^{7b} with R^{7c} is optionally substituted with from one to three members selected from the group consisting of —OH, —OR^{m}, —OC(O)NHR^{m}, —OC(O)N(R^{m})₂, —SH, —SR^{m}, —S(O)R^{m}, —S(O)₂R^{m}, —SO₂NH₂, —S(O)₂NHR^{m}, —S(O)₂N(R^{m})₂, —NHS(O)₂R^{m}, —NR^{m}S(O)₂R^{m}, —C(O)NH₂, —C(O)NHR^{m}, —C(O)N(R^{m})₂, —C(O)R^{m}, —NHC(O)R^{m}, —NR^{m}C(O)R^{m}, —NHC(O)NH₂, —NR^{m}C(O)NH₂, —NR^{m}C(O)NHR^{m}, —NHC(O)NHR^{m}, —NR^{m}C(O)N(R^{m})₂, —NHC(O)N(R^{m})₂, —CO₂H, —CO₂R^{m}, —NHCO₂R^{m}, —NR^{m}CO₂R^{m}, —CN, —NO₂, —NH₂, —NHR^{m}, —N(R^{m})₂, —NR^{m}S(O)NH₂ and —NR^{m}S(O)₂NHR^{m}, wherein each R^{m} is independently an unsubstituted C₁₋₆ alkyl.

2. A compound of claim 1, having formula I.

3. A compound of claim 1, having formula II.

4. A compound of claim 1, having formula III.

5. A compound of claim 2, wherein m is 2, n is 1 and p is 0.

6. A compound of claim 2, wherein L is selected from the group consisting of 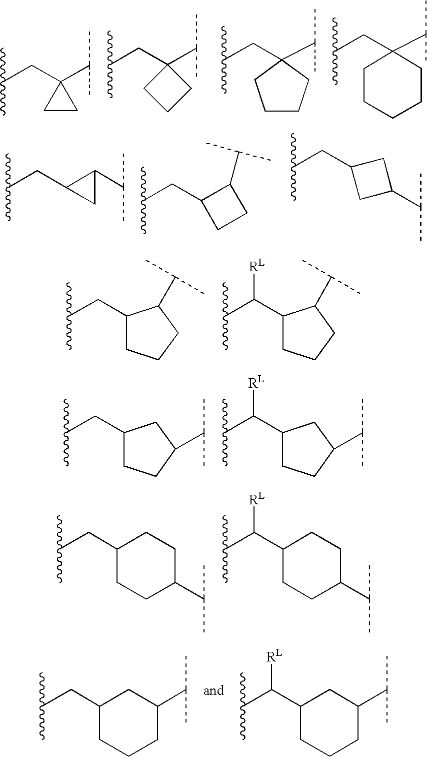
wherein the wavy line indicates the point of attachment to the pyrrolidinyl nitrogen atom, the dashed line indicates the point of attachment to NR⁴R⁵, and R^{L} is a C₁₋₃ alkyl group.

7. A compound of claim 2, wherein R¹ is H or OCH₃; R² and R³ are each independently selected from the group consisting of C₁₋₃ alkyl and C₁₋₃ haloalkyl; R⁴ and R⁵ are each independently selected from the group consisting of H, C₁₋₄ alkyl, C₁₋₄ haloalkyl, C₃₋₆ cycloalkyl, —COR^{a}, and —SO₂R^{a}; R⁶ is H or CH₃; and each R⁸ when present is independently selected from the group consisting of halogen and C₁₋₄ alkyl.

8. A compound of claim 6, wherein L is a member selected from the group consisting of

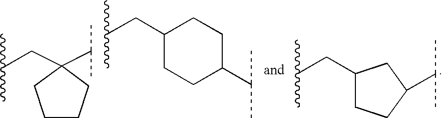

9. A compound of claim 8, wherein R¹ is H or OCH₃; R² and R³ are each independently selected from the group consisting of C₁₋₃ alkyl and C₁₋₃ haloalkyl; R⁴ and R⁵ are each independently selected from the group consisting of H, C₁₋₄ alkyl, C₁₋₄ haloalkyl, C₃₋₆ cycloalkyl, —COR^{a}, and —SO₂R^{a}; R⁶ is H or CH₃; and each R⁸ when present is independently selected from the group consisting of halogen and C₁₋₄ alkyl.

10. A compound of claim 3, wherein R^{7b} and R^{7c} are combined to form a five or six-membered ring fused to the pyrrolidine ring.

11. A compound of claim 3, wherein R^{7a} is selected from the group consisting of hydrogen and C₁₋₈ alkyl.

12. A compound of claim 3, wherein n is 1 or 2.

13. A compound of claim 3, wherein R¹ is selected from the group consisting of hydrogen and C₁₋₈ alkoxy.

14. A compound of claim 3, wherein R² and R³ are each independently selected from the group consisting of methyl, ethyl, propyl, isopropyl, butyl, sec-butyl, isobutyl, tert-butyl and C₁₋₄ haloalkyl.

15. A compound of claim 3, wherein n is 1 or 2; R¹ is selected from the group consisting of hydrogen and C₁₋₈ alkoxy; R² and R³ are each independently selected from the group consisting of methyl, ethyl, propyl, isopropyl, butyl, sec-butyl, isobutyl, tert-butyl and C₁₋₄ haloalkyl.

16. A compound of claim 10, wherein n is 1 or 2; R¹ is selected from the group consisting of hydrogen and C₁₋₈ alkoxy; and R² and R³ are each independently selected from the group consisting of methyl, ethyl, propyl, isopropyl, butyl, sec-butyl, isobutyl, tert-butyl and C₁₋₄ haloalkyl.

17. A compound of claim 11, wherein n is 1 or 2; R¹ is selected from the group consisting of hydrogen and C₁₋₈ alkoxy; and R² and R³ are each independently selected from the group consisting of methyl, ethyl, propyl, isopropyl, butyl, sec-butyl, isobutyl, tert-butyl and C₁₋₄ haloalkyl.

18. A compound of claim 4, wherein n is 1 or 2.

19. A compound of claim 4, wherein R¹ is selected from the group consisting of hydrogen and C₁₋₈ alkoxy.

20. A compound of claim 4, wherein R² and R³ are each independently selected from the group consisting of methyl, ethyl, propyl, isopropyl, butyl, sec-butyl, isobutyl, tert-butyl and C₁₋₄ haloalkyl.

21. A compound of claim 4, wherein n is 1 or 2; R¹ is selected from the group consisting of hydrogen and C₁₋₈ alkoxy; R² and R³ are each independently selected from the group consisting of methyl, ethyl, propyl, isopropyl, butyl, sec-butyl, isobutyl, tert-butyl and C₁₋₄ haloalkyl.

22. A compound of claim 4, wherein m is 1 or 2 and each R⁸ is independently selected from the group consisting of halogen and C₁₋₈ alkyl.

23. A compound of claim 4, wherein R⁶ is H or CH₃.

24. A compound of claim 4, wherein n is 1 or 2; R¹ is selected from the group consisting of hydrogen and C₁₋₈ alkoxy; R² and R³ are each independently selected from the group consisting of methyl, ethyl, propyl, isopropyl, butyl, sec-butyl, isobutyl, tert-butyl and C₁₋₄ haloalkyl; R⁶ is H or CH₃; m is 1 or 2 and each R⁸ is independently selected from the group consisting of halogen and C₁₋₈ alkyl.

25. A compound of claim 1, wherein said compound is selected from the group consisting of compounds 1-20 in Table B.

26. A pharmaceutical composition comprising a compound of claim 1, and a pharmaceutically acceptable excipient.

27-37. (canceled)
